(19) United States
(12) Patent Application Publication
(10) Pub. No.: US 2017/0252304 A1
(43) Pub. Date: Sep. 7, 2017

(54) Encapsulation Methods and Compositions

(71) Applicant: Prodo Laboratories, Inc., Aliso Viejo, CA (US)

(72) Inventors: David Scharp, Aliso Viejo, CA (US); Roy McCord, Huntington Beach, CA (US)

(21) Appl. No.: 15/446,729

(22) Filed: Mar. 1, 2017

Related U.S. Application Data
(60) Provisional application No. 62/302,167, filed on Mar. 1, 2016.

(51) Int. Cl. A61K 9/50; A61K 49/04; A61K 35/39

(52) U.S. Cl. CPC A61K 9/5031; A61K 35/39; A61K 49/0419

(57) ABSTRACT

This invention provides methods for the formation of biocompatible membranes around biological materials using photopolymerization of water soluble molecules. The membranes can be used as a covering to encapsulate biological materials or biomedical devices, as a “glue” to cause more than one biological substance to adhere together, or as carriers for biologically active species. Several methods for forming these membranes are provided. Each of these methods utilizes a polymerization system containing water-soluble macromers, species, which are at once polymers and macromolecules capable of further polymerization. The macromers are polymerized using a photoinitiator (such as a dye), optionally a cocatalyst, optionally an accelerator, and radiation in the form of visible or long wavelength UV light. The reaction occurs either by suspension polymerization or by interfacial polymerization. The polymer membrane can be formed directly on the surface of the biological material, or it can be formed on material, which is already encapsulated.

### RELATED APPLICATIONS

This application is a Non-provisional application of U.S. provisional application Ser. No. 62/302,167 filed on Mar. 1, 2016, the contents of which are all herein incorporated by this reference in their entireties. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### FIELD OF THE INVENTION

The present invention relates to compositions and methods of encapsulation of biological material into a patient in need of treatment.

### BACKGROUND OF THE INVENTION

Microencapsulation technology holds promise in many areas of medicine. For example, some important applications are treatment of diabetes, production of biologically important chemicals, evaluation of anti-human immunodeficiency virus drugs, encapsulation of hemoglobin for red blood cell substitutes, and controlled release of drugs. During encapsulation using prior methods, cells are often exposed to processing conditions, which are potentially cytotoxic. These conditions include heat, organic solvents and non-physiological pH, which can kill or functionally impair cells. Proteins are often exposed to conditions that are potentially denaturing and can result in loss of biological activity.

Further, even if cells survive processing conditions, the stringent requirements of encapsulating polymers for biocompatibility, chemical stability, immunoprotection and resistance to cellular overgrowth, restrict the applicability of prior art methods. For example, the encapsulating method based on ionic crosslinking of alginate (a polyanion) with polylysine or polyomithine (polycation) offers relatively mild encapsulating conditions, but the long-term mechanical and chemical stability of such ionically crosslinked polymers remains doubtful. Moreover, these polymers when implanted in vivo, are susceptible to cellular overgrowth, which restricts the permeability of the microcapsule to nutrients, metabolites, and transport proteins from the surroundings. This has been seen to possibly lead to starvation and death of encapsulated islets of Langerhans cells.

Thus, there is a need for a relatively mild cell encapsulation method, which offers control over properties of the encapsulating polymer. The membranes must be non-toxically produced in the presence of cells, with the qualities of being permselective, chemically stable, and very highly biocompatible. A similar need exists for the encapsulation of biological materials other than cells and tissues.

### Biocompatibility

Synthetic or natural materials intended to come in contact with biological fluids or tissues are broadly classified as biomaterials. These biomaterials are considered biocompatible if they produce a minimal or no adverse response in the body. For many uses of biomaterials, it is desirable that the interaction between the physiological environment and the material be minimized. For these uses, the material is considered “biocompatible” if there is minimal cellular growth on its surface subsequent to implantation, minimal inflammatory reaction, and no evidence of anaphylaxis during use. Thus, the material should elicit neither a specific humoral nor cellular immune response, nor a nonspecific foreign body response.

Materials successful in preventing all of the above responses are relatively rare; biocompatibility is more a matter of degree rather than an absolute state. The first event occurring at the interface of any implant with surrounding biological fluids is protein adsorption. In the case of materials of natural origin, it is conceivable that specific antibodies for that material exist in the repertoire of the immune defense mechanism of the host. In this case a strong immune response can result. Most synthetic materials, however, do not elicit such a reaction. They can either activate the complement cascade or adsorb serum proteins that mediate cell adhesion, called cell adhesion molecules (CAMs). The CAM family includes proteins such as fibronectin, vitronectin, laminin, von Willebrand factor, and thrombospondin.

Proteins can adsorb on almost any type of material. They have positively and/or negatively charged regions, as well as hydrophilic and hydrophobic regions. They can thus interact with implanted material through any of these various regions, resulting in cellular proliferation at the implant surface. Complement fragments such as C3b can be immobilized on the implant surface and act as chemoattractants. They in turn can activate inflammatory cells such as macrophages and neutrophils and cause their adherence and activation on the implant. Those cells attempt to degrade and digest the foreign material.

In the event that the implant is nondegradable and is too large to be ingested by large single activated macrophages, the inflammatory cells may undergo frustrated phagocytosis. Several such cells can combine to form foreign body giant cells. In this process, these cells release peroxides, hydrolytic enzymes, and chemoattractant and anaphylactic agents such as interleukins, which increase the severity of the reaction. They also induce the proliferation of fibroblasts on foreign surfaces.

Fibroblasts secrets a collagenous matrix which ultimately results in encasement of the entire implant in a fibrous envelope. Cell adhesion can also be mediated on a charged surface by the cell surface proteoglycans such as heparin sulfate and chondroitin sulfate. In such a process, intermediary CAMs are not required and the cell surface can interact directly with the surface of the implant.

### Enhancing Biocompatibility

Past approaches to enhancing biocompatibility of materials started with attempts at minimization of interfacial energy between the material and its aqueous surroundings. Similar interfacial tensions of the solid and liquid were expected to minimize the driving force for protein adsorption and this was expected to lead to reduced cell adhesion and thrombogenicity of the surface.

Protein adsorption and desorption, however, is a dynamic phenomenon, as seen in the Vroman effect. This effect is the gradual displacement of one serum protein by another, through a well-defined series, until only virtually irreversibly adsorbed proteins are present on the surface. Affinity of protein in a partially dehydrated state for the polymer surface has been proposed as a determining factor for protein adsorption onto a surface. Enhancement of surface hydrophilicity has resulted in mixed success; increased hydrophilicity or hydrophobicity does not have a clear relation with biocompatibility. In some cases, surfaces with intermediate hydrophilicities demonstrate proportionately less protein adsorption. The minimization of protein adsorption may depend both upon hydrophilicity and the absence of change, as described further below, perhaps in addition to other factors.

### Use of Gels in Biomaterials

Gels made of polymers which swell in water such as poly (HEMA), water-insoluble polyacrylates, and agarose, have been shown to be capable of encapsulating islet cells and other animal tissue. However, these gels have undesirable mechanical properties. Agarose forms a weak gel, and the polyacrylates must be precipitated from organic solvents, thus increasing the potential for cytotoxicity. Microencapsulation of islets has been done by polymerization of acrylamide to form polyacrylamide gels. However, the polymerization process, if allowed to proceed rapidly to completion, generates local heat and requires the presence of toxic crosslinkers. This usually results in mechanically weak gels whose immunoprotective ability has not been established. Moreover, the presence of a low molecular weight monomer is required which itself is cytotoxic.

Microcapsules formed by the coacervation of alginate and poly (L-lysine) (PLL) have been shown to be immunoprotective. However, implantation for periods up to a week has resulted in severe fibrous overgrowth on these microcapsules.

### Use of Poly(Ethylene Oxide) (PEO) in Biomaterials

The use of poly(ethylene oxide) (PEO) to increase biocompatibility is well-documented in the literature. The presence of grafted PEO on the surface of bovine serum albumin has been shown to reduce immunogenicity in a rabbit and to increase circulation times of exogenous proteins in animals. The biocompatibility of algin-poly(L-lysine) microcapsules has been significantly enhanced by incorporating a graft copolymer of poly (L-lysine) (PLL) and PEO on the microcapsule surface. The grafting of methoxy PEO onto polyacrylonitrile surfaces was seen to render the polyacrylonitrile surface relatively non-thrombogenic.

PEO is a unique polymer in terms of structure. The PEO chain is highly water soluble and highly flexible. Polymethylene glycol, on the other hand, undergoes rapid hydrolysis, while polypropylene oxide is insoluble in water. PEO chains have an extremely high motility in water and are completely non-ionic in structure. The synthesis and characterization of PEO derivatives which can be used for attachment of PEO to various surfaces, proteins, drugs etc. has been reviewed. Other polymers are also water soluble and non-ionic, such as poly(N-vinyl pyrrolidinone) and poly(ethyl oxazoline). These have been used to reduce interaction of cells with tissues. Water soluble ionic polymers, such as hyaluronic acid, have also been used to reduce cell adhesion to surfaces and can similarly be used.

Immobilization of PEO on a charged surface, such as a coacervated membrane of alginate-PLL, results in shielding of surface charges by the non-ionic PEO. The highly motile PEO chain sweeps out a free volume in its microenvironment. The free volume exclusion effect makes the approach of a macromolecule (viz., a protein) close to a surface which has grafted PEO chains sterically unfavorable. Thus protein adsorption is minimized and cell adhesion is reduced, resulting in surfaces showing increased biocompatibility.

Immobilization of PEO on a surface has been largely carried out by the synthesis of graft copolymers having PEO side chains. This process involves the custom synthesis of monomers and polymers for each application. The use of graft copolymers, however, still does not guarantee that the surface “seen” by a macromolecule consists entirely of PEO.

Electron beam crosslinking has been used to synthesize PEO hydrogels, and these biomaterials have been reported to be non-thrombogenic. However, use of an electron bean precludes the presence of any living tissue due to the sterilizing effect of this radiation. Also, the networks produced are difficult to characterize due to the non-specific crosslinking induced by the electron beam.

Photopolymerizable polyethylene glycol diacrylates have been used to entrap yeast cells for fermentation and chemical conversion. However, yeast cells are widely known to be much hardier, resistant to adverse environments and elevated temperatures, and more difficult to kill when compared to mammalian cells and human tissues. For example, yeast may be grown anaerobically, whereas mammalian cells may not; yeast are more resistant to organic solvents (e.g., ethanol to 12%) than are mammalian cells (e.g., ethanol to <1%); and yeast possess a polysaccharide cell wall, whereas mammalian cells, proteins, polysaccharides, and drugs do not. However, the exposure of sensitive eukaryotic tissue, organisms, or sensitive molecules to the chemical conditions used during polymerization because their polymerization conditions are incompatible with sensitive materials. For example, there are no reports of the encapsulation of mammalian cells using prior art photosensitive prepolymers without a marked loss of cellular function.

Other earlier encapsulations of cells within photopolymerizable materials have focused on microbial cells. Each describes the use of near ultraviolet light (wavelength <320 nm), which is injurious to more sensitive cells such as mammalian cells or higher eukaryotic cells. The technique would be appropriate for microbial cells, but there is no indication of usefulness for more sensitive cells.

Moreover, the prior use of such materials for the entrapment of biological materials is entirely focused on industrial technology, rather than biomedical technology. For example, no attention is paid to biocompatibility, including formulation of the gel to avoid the problems described above. This is an important issue, since bioincompatibility in biomedical applications leads to xenograft failure in therapeutically transplanted cells for the evaluation of drug efficacy and to xenograft failure in diagnostically transplanted cells. Similarly, bioincompatibility would lead to the failure of encapsulated enzymes (for example, therapeutic enzymes encapsulated and circulating or implanted in a blood-rich tissue). Such encapsulated and entrapped enzymes could leave the circulation by interaction with the reticuloendothelial system or could become overgrown with tissues in a foreign body reaction.

Other ways of producing PEO hydrogels include use of PEO chains end capped with n-alkane chains, which associate in aqueous media to form stable gels. No biological properties of these materials have been reported. Thus, the prior art contains no description of methods to form biocompatible PEO networks on three-dimensional living tissue surfaces without damaging encapsulated tissue.

Among the techniques for encapsulating mammalian tissue with polymers other than PEO is a method of photopolymerizing the monomer 2-hydroxyethyl methacrylate (“HEMA”) and the crosslinking agent ethylene glycol dimethacrylate (“EGDA”) in a cylindrical mold containing the biological material. The product of this reaction, a cylindrical gel with cells embedded throughout, is frozen and then finely ground into small particles. This technique, however, suffers from a number of disadvantages. First, because the cylindrical gel is broken along random planes, shearing will often occur through pockets of cells, leaving some cells exposed to the host immune system. Second, HEMA and EGDA are small cytotoxic molecules capable of penetrating the cellular membrane. Third, the resulting polymer membrane has uneven pore sizes, which vary to an upper limit of 20 microns, thereby allowing transit of immune response molecules. These drawbacks are reflected in data, which show that tissue remains viable for only 2-3 days after this encapsulation process.

Encapsulation of human cells, specifically human islets, is seen as a way to treat Diabetes mellitus. Diabetes mellitus is a disease caused by the loss of the ability to transport glucose into the cells of the body, because of either a lack insulin production or diminished insulin response. In a healthy person, minute elevations in blood glucose stimulate the production and secretion of insulin, the role of which is to increase glucose uptake into cells, returning the blood glucose to the optimal level. Insulin stimulates liver and skeletal muscle cells to take up glucose from the blood and convert it into glycogen, an energy storage molecule. It also stimulates skeletal muscle fibers to take up amino acids from the blood and convert them into protein, and it acts on adipose (fat) cells to stimulate the synthesis of fat. In diabetes, glucose saturates the blood stream, but it cannot be transported into the cells where it is needed and utilized. As a result, the cells of the body are starved of needed energy, which leads to the wasted appearance of many patients with poorly controlled insulin-dependent diabetes.

Prior to the discovery of insulin and its use as a treatment for diabetes, the only available treatment was starvation followed predictably by death. Death still occurs today with insulin treatment from over dosage of insulin, which results in extreme hypoglycemia and coma followed by death unless reversed by someone who can quickly get glucose into the patient. Also, death still occurs from major under dosage of insulin, which leads to hyperglycemia and ketoacidosis that can result in coma and death if not properly and urgently treated.

While diabetes is not commonly a fatal disease thanks to the treatments available to diabetics today, none of the standard treatments can replace the body's minute-to-minute production of insulin and precise control of glucose metabolism. Therefore, the average blood glucose levels in diabetics generally remain too high. The chronically elevated blood glucose levels cause a number of long-term complications. Diabetes is the leading cause of new blindness, renal failure, premature development of heart disease or stroke, gangrene and amputation, and impotence. It decreases the sufferer's overall life expectancy by one to two decades.

Diabetes mellitus is one of the most common chronic diseases in the world. In the United States, diabetes affects approximately 16 million people—more than 12% of the adult population over 45. The number of new cases is increasing by about 150,000 per year. In addition to those with clinical diabetes, there are approximately 20 million people showing symptoms of abnormal glucose tolerance. These people are borderline diabetics, midway between those who are normal and those who are clearly diabetic. Many of them will develop diabetes in time and some estimates of the potential number of diabetics are as high as 36 million or 25-30% of the adult population over 45 years.

Diabetes and its complications have a major socioeconomic impact on modem society. Of the approximately $700 billion dollars spent on healthcare in the US today, roughly $100 billion is spent to treat diabetes and its complications. Since the incidence of diabetes is rising, the costs of diabetes care will occupy an ever-increasing fraction of total healthcare expenditures unless steps are taken promptly to meet the challenge. The medical, emotional and financial toll of diabetes is enormous, and increase as the numbers of those suffering from diabetes grows.

Diabetes mellitus can be subdivided into two distinct types: Type 1 diabetes and Type 2 diabetes. Type 1 diabetes is characterized by little or no circulating insulin, and it most commonly appears in childhood or early adolescence. There is a genetic predisposition for Type 1 diabetes. It is caused by the destruction of the insulin-producing beta cells in the islets of Langerhans; which are scattered throughout the pancreas, an elongated gland located transversely behind the stomach. The beta cells are attacked by an autoimmune reaction initiated by some as yet unidentified environmental event. Possibly a viral infection or noninfectious agent (a toxin or a food) triggers the immune system to react to and destroy the patient's beta cells in the pancreas. The pathogenic sequence of events leading to Type 1 diabetes is thought to consist of several steps. First, it is believed that genetic susceptibility is an underlying requirement for the initiation of the pathogenic process. Secondly, an environmental insult mediated by a virus or noninfectious pathogen in food triggers the third step, the inflammatory response in the pancreatic islets (insulitis). The fourth step is an alteration or transformation of the beta cells such that they are no longer recognized as “self” by the immune system, but rather seen as foreign cells or “nonself”. The last step is the development of a full-blown immune response directed against the “targeted” beta cells, during which cell-mediated immune mechanisms cooperate with cytotoxic antibodies in the destruction of the insulin-producing beta cells. Despite this immune attack, for a period, the production of new beta cells is fast enough to stay ahead of the destruction by the immune system and a sufficient number of beta cells are present to control blood glucose levels. However, the number of beta cells gradually declines. When the number of beta cells drops to a critical level (10% of normal), blood glucose levels no longer can be controlled and progression to total insulin production failure is almost inevitable. It is thought that the regeneration of beta cells continues for a few years, even after functional insulin production ceases, but that the cells are destroyed as they develop to maturity.

To reduce their susceptibility to both the acute and chronic complications of diabetes, people with Type 1 diabetes must take multiple insulin injections daily and test their blood sugar multiple times per day by pricking their fingers for blood. They then have to decide how much insulin to take based on the food eaten and level of physical activity, amount of stress, and existence of any illness over the next few hours. The multiple daily injections of insulin do not adequately mimic the body's minute-to-minute production of insulin and precise control of glucose metabolism. Blood sugar levels are usually higher than normal, causing complications that include blindness, heart attack, kidney failure, stroke, nerve damage, and amputations. Even with insulin, the average life expectancy of a diabetic is 15-20 years less than a healthy person.

Type 2 diabetes usually appears in middle age or later, and particularly affects those who are overweight. Over the past few years, however, the incidence of Type 2 diabetes mellitus in young adults has increased dramatically. In the last several years, the age of onset for Type 2 diabetes in obese people has dropped from 40 years to 30 years. These are the new younger victims of this disease. In Type 2 diabetes, the body's cells that normally require insulin lose their sensitivity and fail to respond to insulin normally. This insulin resistance may be overcome for many years by extra insulin production by the pancreatic beta cells. Eventually, however, the beta cells are gradually exhausted because they have to produce large amounts of excess insulin due to the elevated blood glucose levels. Ultimately, the overworked beta cells die and insulin secretion fails, bringing with it a concomitant rise in blood glucose to sufficient levels that it can only be controlled by exogenous insulin injections. High blood pressure and abnormal cholesterol levels usually accompany Type 2 diabetes. These conditions, together with high blood sugar, increase the risk of heart attack, stroke, and circulatory blockages in the legs leading to amputation. Drugs to treat Type 2 diabetes include some that act to reduce glucose absorption from the gut or glucose production by the liver, others that reduce the formation of more glucose by the liver and muscle cells, and others that stimulate the beta cells directly to produce more insulin. However, high levels of glucose are toxic to beta cells, causing a progressive decline of function and cell death. Consequently, many patients with Type 2 diabetes eventually need exogenous insulin.

Another form of diabetes is called Maturity Onset Diabetes of the Young (MODY). This form of diabetes is due to one of several genetic errors in insulin-producing cells that restrict their ability to process the glucose that enters via special glucose receptors. Beta cells in patients with MODY cannot produce insulin correctly in response to glucose, which results in hyperglycemia. The patients treatment eventually leads to the requirement for insulin injections.

The currently available medical treatments for insulin-dependent diabetes are limited to insulin administration and pancreas transplantation with either whole pancreata or pancreatic segments.

Insulin therapy is by far more prevalent than pancreas transplantation. Insulin administration is conventionally either by a few blood glucose measurements and subcutaneous injections, intensively by multiple blood glucose measurements and through multiple subcutaneous injections of insulin, or by continuous subcutaneous injections of insulin with a pump. Conventional insulin therapy involves the administration of one or two injections a day of intermediate-acting insulin with or without the addition of small amounts of regular insulin. The intensive insulin therapy involves multiple administration of intermediate- or long-acting insulin throughout the day together with regular or short-acting insulin prior to each meal. Continuous subcutaneous insulin infusion involves the use of a small battery-driven pump that delivers insulin subcutaneously to the abdominal wall, usually through a 27-gauge butterfly needle. This treatment modality has insulin delivered at a basal rate continuously throughout the day and night, with increased rates programmed prior to meals. In each of these methods, the patient is required to frequently monitor his or her blood glucose levels and, if necessary, adjust the insulin dose. However, controlling blood sugar is not simple. Despite rigorous attention to maintaining a healthy diet, exercise regimen, and always injecting the proper amount of insulin, many other factors can adversely affect a person's blood-sugar including stress, hormonal changes, periods of growth, illness, infection and fatigue. People with Type 1 diabetes must constantly be prepared for life threatening hypoglycemic (low blood sugar) and hyperglycemic (high blood sugar) reactions. Insulin-dependent diabetes is a life threatening disease, which requires never-ending vigilance.

In contrast to insulin administration, whole pancreas transplantation or transplantation of segments of the pancreas is known to eliminate the elevated glucose values by regulating insulin release from the new pancreas in diabetic patients. Histologically, the pancreas is composed of three types of functional cells; a) exocrine cells that secrete their enzymes into a small duct, b) ductal cells that carry the enzymes to the gut, and c) endocrine cells that secrete their hormones into the bloodstream. The exocrine portion is organized into numerous small glands (acini) containing columnar to pyramidal epithelial cells known as acinar cells. Acinar cells comprise approximately 80% of the pancreatic cells and secrete into the pancreatic duct system digestive enzymes, such as, amylases, lipases, phospholipases, trypsin, chymotrypsin, aminopeptidases, elastase and various other proteins. Approximately 1.5 and 3 liters of alkaline fluid are released per day into the common bile duct to aid digestion.

The pancreatic duct system consists of an intricate, tributary-like network of interconnecting ducts that drain each secretory acinus, draining into progressively larger ducts, and ultimately draining into the main pancreatic duct. The lining epithelium of the pancreatic duct system consists of duct cells. Approximately 10% of the pancreas cells are duct cells. Duct cell morphology ranges from cuboidal in the fine radicles draining the secretory acini to tall, columnar, mucus secreting cells in the main ductal system.

Hormone producing islets are scattered throughout the pancreas and secrete their hormones into the bloodstream, rather than ducts. Islets are richly vascularized. Islets comprise only 1-2% of the pancreas, but receive about 10 to 15% of the pancreatic blood flow. There are three major cell types in the islets, each of which produces a different endocrine product: alpha cells secrete the hormone glucagon (glucose release); beta cells produce insulin (glucose use and storage) and are the most abundant of the islet cells; and delta cells secrete the hormone somatostatin (inhibits release of other hormones). These cell types are not randomly distributed within an islet. The beta cells are located in the central portion of the islet and are surrounded by an outer layer of alpha and delta cells. Besides insulin, glucagon and somatostatin, gastrin and Vasoactive Intestinal Peptide (VIP) have been identified as products of pancreatic islets cells.

Pancreas transplantation is usually only performed when kidney transplantation is required, which makes pancreas-only transplantations relatively infrequent operations. Although pancreas transplants are very successful in helping people with insulin-dependent diabetes improve their blood sugar control without the need for insulin injections and reduce their long-term complications, there are a number of drawbacks to whole pancreas transplants. Most importantly, getting a pancreas transplant involves a major operation and requires the use of life-long immune suppressant drugs to prevent the body's immune system from destroying the pancreas. The pancreas is destroyed in a manner of days without these drugs. Some risks in taking these immuno-suppressive drugs are the increased incidence of infections and tumors that can be life threatening in their own right. The risks inherent in the operative procedure, the requirement for life-long immunosuppression of the patient to prevent rejection of the transplant, and the morbidity and mortality rate associated with this invasive procedure, illustrate the serious disadvantages associated with whole pancreas transplantation for the treatment of diabetes. Thus, an alternative to insulin injections or pancreas transplantation would fulfill a great public health need.

Islet transplants are much simpler (and safer) procedures than whole pancreas transplants and can achieve the same effect by replacing the destroyed beta cells. As discussed above, when there are insufficient numbers of beta cells, or insufficient insulin secretion, regardless of the underlying reason, diabetes results. Reconstituting the islet beta cells in a diabetic patient to a number sufficient to restore normal glucose-responsive insulin production would solve the problems associated with both insulin injection and major organ transplantation. Microencapsulation and implantation of islet cells into diabetic patients holds promise for treatment of those with diabetes.

Encapsulation of cells for the potential of treating a number of diseases and disorders has been discussed in the literature. The concept was suggested as early as 100 years ago, but little work was done prior to the 1950's when immunologists began using encapsulated cells with membrane devices to separate the cells from the host to better understand the different aspects of the immune system. Research on implantation was underway in the 1970's and 1980's with the first review written in 1984. Several additional reviews have been written since then explaining the different approaches and types of devices under development. Cell encapsulation technology has potential applications in many areas of medicine. For example, some important potential applications are treatment of diabetes, production of biologically important chemicals, and evaluation of anti-human immunodeficiency virus drugs.

There are several types of encapsulated devices. Macrodevices are larger devices containing membranes in the form of sheets or tubes for permselectivity and usually supporting structures. They contain one or several compartments for the encapsulated cells. They are designed for implantation into extravascular or vascular sites. Some are designed to grow into the host to increase oxygen diffusion into these large devices. Others are designed to have no reaction by the host, thus increasing their ease of removal from different sites. There have been two major types of macrodevices developed: a] flat sheet and b] hollow fiber.

Among the flat sheet devices, one type (Baxter, Theracyte) is made of several layers for strength and has diffusion membranes between support structures with loading ports for replacing the cells. The other type is more simple in design. The device uses alginate based membranes and other supporting membranes to encapsulate islets within an alginate matrix between the sheets. The complex device is designed to grow into the body to increase diffusion of oxygen. Due to its relatively large size, there are few sites in the body able to accommodate it for the treatment of a disease like diabetes. Since it grows into the body and the contained cells are not expected to survive for more than a few years, multiple cell removals and reloading of new cells is required for the long-term application of this device. It has proven quite difficult to flush and reload this type of device while at the same time maintaining the critical cell compartment distance for oxygen diffusion.

The second flat sheet style of device is designed to be an “all in/all out” device with little interaction with the host. For the diabetes product, it has been quite difficult to place this device into the intraperitoneal cavity of large animals, while maintaining its integrity. This has been due to the difficulty in securing it in the abdomen so that the intestines cannot cause it to move or wrinkle, which may damage or break the device.

The other major macrodevice type is the hollow fiber, made by extruding thermoplastic materials into hollow fibers. These hollow fibers can be made large enough to act as blood conduits. One model is designed to be fastened into the host's large blood vessels and the encapsulated cells are behind a permselective membrane within the device. This type has shown efficacy in large animal diabetic trials, but has been plagued by problems in the access to the vascular site. Both thrombosis and hemorrhage have complicated the development of this approach with it currently being abandoned as a clinically relevant product. Another model using hollow fibers is much smaller in diameter and designed to be used as an extravascular device. Due to low packing densities, the required cell mass for encapsulation causes the length of this type of hollow to approach many meters. Therefore, this approach was abandoned for treating diabetes since it was not clinically relevant. In addition, sealing the open ends of the fiber is not trivial and strength has been a problem depending upon the extravascular site.

The microcapsule was one of the first to offer potential clinical efficacy. Alginate microcapsules were used to encapsulate islets, which eliminated diabetes in rodents when implanted intraperitoneally. However, nearly 25 years have passed since these first reports without the ability to demonstrate clinical efficacy. One of the problems associated with microcapsules is their relatively large size in combination with low packing densities of cells, especially for the treatment of diabetes. Another is the use of alginate; an ionically crosslinked hydrogel dependent upon the calcium concentration for its degree of crosslinking. The permselectivity of pure alginate capsules has been difficult to control with the vast majority being wide open in terms of molecular weight cutoff. Varieties of positively charged crosslinked agents, such as polylysine, have been added as a second coating to provide permselectivity to the capsule. However, polylysine and most other similar molecules invite an inflammatory reaction requiring an additional third coating of alginate to reduce the host's response to the capsule. In addition, it has been difficult to produce very pure alginates that are not reactive within the host after implantation. Trying to reduce the size of the alginate microcapsules causes two major problems. First, the production of very large quantities of empty capsules without any cells. Second, the formation of smaller capsules results in poorly coated cells. There is no force to keep the contained cells within the center of the microcapsule, which causes the risk of incomplete coatings to go up exponentially with the decrease in the size of the capsules.

Another form cell encapsulation is micro-bulk coating. A micro-bulk coated cell aggregate is one that has a cell coating around the cell aggregate regardless of size or shape of the aggregate. Furthermore, it has strength and stability, thus preventing the coated material from being violated by the host's immune system.

An important aspect to the feasibility of using these various methods is the relevant size and implant site needed to obtain a physiological result of 15,000 IEQ/kg-BW. Injecting isolated islets into the Portal Vein requires 2-3 ml of pack cells. A macro-device consisting of a flat sheet that is 1 islet thick (˜500 μm) requires a surface area equivalent to 2 US dollar bills. A macro-device consisting of hollow fibers with a loading density of 5% would need 30 meters of fiber. Alginate microcapsules with an average diameter of 400-600 μm would need a volume of 50-170 ml. However, PEG micro-bulk coating of islets which produces a 25-50 μm thick covering would only need a volume of 6-12 ml and could be injected into almost any area in the body.

Hubbell et al. (U.S. Pat. No. 5,529,914 and related patents) disclose methods for the formation of biocompatible membranes around biological materials using photopolymerization of water-soluble molecules. Each of these methods utilizes a polymerization system containing water-soluble macromers, polymerization using a photoinitiator (such as a dye), and radiation in the form of visible or long wavelength UV light.

Due to the inability of those of skill in the art to provide one or more important properties of successful cell encapsulation, none of the encapsulation technologies developed in the past have resulted in a clinical product. These properties can be broken down into the following categories:

Biocompatibility—The materials used to make an encapsulating device must not elicit a host response, which may cause a non-specific activation of the immune system by these materials alone. When considering immunoisolation, one must recognize that it will only work in the situation where there is no activation of the host immune cells to the materials. If there is activation of the host immune cells by the materials, then the responding immune cells will surround the device and attempt to destroy it. This process produces many cytokines that will certainly diffuse through the capsule and most likely destroy the encapsulated cells. Most devices tested to date have failed in part by their lack of biocompatibility in the host.

Permselectivity—There exists an important balance between having the largest pores as possible in the capsule surrounding the encapsulated cells to permit all the nutrients and waste products to pass through the capsule to permit optimal survival and function, while at the same time, the smallest pore size as possible in the capsule to keep all elements of the immune system away from the encapsulated cells to prevent degradation of the cells. Small pores capable of keeping out immune cytokines also cause the death of the encapsulated cells from a lack of diffusion of nutritional elements and waste products. The optimal cell encapsulation has an exact and consistent permselectivity, which allows maximal cell survival and function, as well as, provides isolation from the host immune response. Ideally, this encapsulation technology should offer the ability to select and change the pore size as required by the encapsulated cells and their function, as well as pore size variation based on whether the cells are allograft or xenograft cells.

Encapsulated Cell Viability and Function—The encapsulating materials should not exhibit cytotoxicity to the encapsulated cells either during the formation of the coatings or on an ongoing basis, otherwise the number of encapsulated cells will decrease and risk falling short of the number required for a therapeutically effective treatment of a disease or disorder.

Relevant Size—Many devices are of such a large size that the number of practical implantation sites in the host is limited. Another factor is the relative diffusion distance between the encapsulated cells and the host. The most critical diffusive agent for cell survival is oxygen. These diffusion distances should be minimal since the starting partial pressure of oxygen is in the range of 30-40 mm Hg at the tissue level in the body. There is little tolerance for a reduction in diffusive distances, due to the initially low oxygen partial pressure. This would further lower the oxygen concentration to a point where the cells cannot adequately function or survive.

Cell Retrieval or Replacement—The encapsulating device should be retrievable, refillable, or biodegradable, allowing for replacement or replenishment of the cells. Many device designs have not considered the fact that encapsulated cells have a limited lifetime in the host and require regular replacement.

Therapeutic Effect—The implant should contain sufficient numbers of functional cells to have a therapeutic effect for the disease application in the host.

Clinical Relevance—The encapsulating cell device should have a total volume or size that allows it to be implanted in the least invasive or most physiologic site for function, which has a risk/benefit ratio below that faced by the host with the current disease or disorder.

Commercial Relevance—The encapsulating cell device should be able to meet the above requirements in order for it to be produced on an ongoing basis for the long-term treatment of the disease process for which it has been designed.

All of the above factors must be taken into consideration when evaluating a specific technique, method or product for use in implantation of islets to alleviate the effects of diabetes.

Transplantation of human islets with immunosuppression is done by injecting unencapsulated islets into the portal vein by direct injection percutaneously between the ribs, into the liver, and then the portal vein by fluoroscopic direction. Essentially all of the human islet transplants have been done by this technique, except for the first ones done by umbilical vein injection via a cutdown. A major risk of this procedure is the fact that injection of islets into the portal vein leads to increased portal venous pressures depending on the rate of infusion and the amount infused. Another risk has been elevated portal venous pressures from large volumes of injected islet tissues that are not sufficiently purified. This also leads to portal venous thrombosis as a complication of this procedure. As the interventional radiologist prepares to withdraw the catheter, a bolus of gelatin is left behind to prevent hemorrhaging from the injection site. Unfortunately, several patients have had bleeding episodes following this procedure.

In addition to injecting the islets into the portal vein, a few patients have had their islets injected into the body of the spleen. The spleen is more fragile than the liver so these injections were performed at the time of kidney transplantation at which time the splenic injection could be done as an open procedure. Freely injecting the islets into the peritoneal cavity has been performed in mouse transplants without difficulty. In using this site in larger animals or humans, it has been found that twice the number of islets is needed in the peritoneal cavity than required in the portal vein implants. If any rejection or inflammatory reactions occur, then adhesions tend to form between the loops of intestine, as well as, to the omentum. This reaction can lead to additional problems long term, such as, bowel obstruction. Thus, the ability to perform encapsulated islet implants into the subcutaneous site would significantly reduce the complications associated with these other sites.

Attempts at subcutaneous implantation of encapsulated islets have been unable to produce sustainable results in the treatment of diabetes, probably due to some or all of the scientific challenges described above.

### Definitions

As used in the present application, the following definitions apply:

Allografts—grafts between two or more individuals with different HLA or BLC immune antigen makeup at one or more loci (usually with reference to histocompatibility loci).

Athymic mice—has an incomplete immune system.

Autograft—graft taken from one part of the body and returned to the same individual.

ApoE2—a protein that shuttles lipids through the body.

Biocompatibility—the ability to exist alongside living things without harming them.

Cell aggregate—a collection of cells into a mass, unit, or an organelle that are held together by connecting substances, matrices, or structures.

Clinically relevant and Clinical relevance—encapsulating cell or tissue device must be of such a total volume or size to be implantable in the least invasive or most physiologic site for function with the risk/benefit ratio below that of what the host with the disease or disorder faces with the current disease or disorder.

CMRL (Connaught Medical Research Labs) media—well suited for growth of cloning monkey kidney cell cultures and for growth of other mammalian cell lines when enriched with horse or calf serum. Particularly rich in nucleosides and some vitamins.

Commercially relevant and Commercial relevance—encapsulating cell device must be able to meet requirements such as biocompatibility, permselectivity, encapsulated cell viability and function, size, cell retrieval or replacement, and therapeutic effect, in order for it to be produced on an ongoing basis for treatment of the disease process for which it has been designed within the acceptance as a product that is successful in the market place.

Conformal Coating—a relatively thin polymer coating that conforms to the shape and size of the coated particle.

C-peptide—the polypeptide chain in proinsulin linking the alpha and beta chains of active insulin. Insulin is initially synthesized in the form of proinsulin. There is one molecule of C-peptide for every molecule of insulin in the blood. C-peptide levels in the blood can be measured and used as an indicator of insulin production when exogenous insulin (from injection) is present and mixed with endogenous insulin (produced by the body). The C-peptide test can also be used to help assess if high blood glucose is due to reduced insulin production or to reduced glucose intake by the cells. Type 1 diabetics have little or no C-peptide in the blood, while Type 2 diabetics can have reduced or normal C-peptide levels. The concentration of C-peptide in non-diabetics is 0.5-3.0 ng/ml.

Cynomolgus primate—crab-eating macaque, Macaca fascicularis, is native to Southeast Asia.

Cytodex beads—microcarrier beads of Dextran with positive-charged trimethyl-2-hydroxyaminopropyl groups on the surface.

Dendrimer—an artificially manufactured or synthesized polymer molecule built up from branched units called monomers. Defined by regular, highly branched monomers leading to a monodisperse, tree-like or generational structure. Synthesized through stepwise reactions, building the dendrimer up one monomer layer, or “generation,” at a time. Each dendrimer consists of a multifunctional core molecule with a dendritic wedge attached to each functional site. The core molecule is referred to as “generation 0.” Each successive repeat unit along all branches forms the next generation, “generation 1,” “generation 2,” and so on until the terminating generation.

Diabetes—a variable disorder of carbohydrate metabolism caused by a combination of hereditary and environmental factors and usually characterized by inadequate secretion or utilization of insulin, by excessive urine production, by excessive amounts of sugar in the blood and urine, and by thirst, hunger, and loss of weight

DTZ (diphenylthiocarbazone)—a dye which binds to the zinc within insulin granules

Eosin Y—C₂₀H.₆O₅Br₄Na₂ [MW 691.914] a red dye soluble in water (40%) and strongly fluorescent. Structure is similar to Eosin Y ws, Ethyl eosin, Eosin B, Phloxine, Erythrosin B, Fluorescein, Rose bengal, and Mercurochrome.

Evan's blue staining—An azo dye used in blood volume and cardiac output measurement by the dye dilution method. It is very soluble, strongly bound to plasma albumin, and disappears very slowly.

Ficoll™—high molecular weight sucrose-polymers used to separate cells.

FDA/EB (fluorescein diacetate/ethidium bromide) staining—When stained, the live cells show up as green colored cells, whereas the cells with cytotoxicity and those with compromised cell membrane functions show red coloration of the nuclei.

“Good” buffer—group of buffers developed by N. E. Good and S. Izawa (Hydrogen ion buffers, Methods Enzymol (1972) 24, 53-68).

HbA1c test [equivalent to Hemoglobin A1C; Glycated hemoglobin]—Test used to assess long-term glucose control in diabetes. Alternative names for this test include glycosylated hemoglobin or Hgb, hemoglobin glycated or glycosylated protein, and fructosamine. HbA1c refers to total glycosylated hemoglobin present in erythrocytes. Due to the fact that glucose stays attached for the life of the cell (about 3 months), the test shows what the person's average blood glucose level over a period of 4-8 weeks. This is a more appropriate test for monitoring a patient who is capable of maintaining long-term, stable control. Test results are expressed as a percentage, with 4 to 6% considered normal. The HbA1c “big picture” complements the day to day “snapshots” obtained from the self-monitoring of blood glucose (mg/dL), and the two tests can be related with the conversion equation: HbA1c=(Plasma Blood Glucose+77.3)/35.6. Glycated protein in serum/plasma assesses glycemic control over a period of 1-2 weeks. A below normal test value is helpful in establishing the patient's hypoglycemic state in those conditions.

HEMA (2-hydroxyethyl methacrylate)—used in light curing polymer system and high performance coatings for lasting high gloss against scratching, solvents and weathering. It is used in crosslinkable paint resins and emulsions, binders for textiles and paper. It is used as a adhesion promoter for metal coatings.

IBMX—A potent cyclic nucleotide phosphodiesterase inhibitor; due to this action, the compound increases cyclic AMP and cyclic GMP in tissue and thereby activates multiple cell processes.

IP (Intraperitoneal)—Within the peritoneal cavity, the area that contains the abdominal organs.

IEQ (Islet equivalent)—definition based on both insulin content and morphology/size. An insulin granule binding dye, such as diphenylthiocarbazone (DTZ) is commonly used to identify beta cells. Since beta cells are only one of several other cell types needed to constitute an islet, a morphological assessment, based upon a mean diameter of 150 μm, is used in addition to staining by DTZ, to define an islet equivalent.

M199—originally formulated for nutritional studies of chick embryo fibroblasts. Contains Earle's salts, L-glutamine, and 2,200 mg/L sodium bicarbonate.

Maturity Onset Diabetes of the Young (MODY).—A form of diabetes characterized by early age of onset (usually less than 25 years of age), autosomal dominant inheritance (that is, it is inherited by 50% of a parent's children) with diabetes in at least 2 generations of the patient's family. MODY diabetes that can often be controlled with meal planning or diabetes pills, at least in the early stages of diabetes. It differs from type 2 diabetes in that patients have a defect in insulin secretion or glucose metabolism, and are not resistant to insulin. MODY accounts for about 2% of diabetes worldwide and 6 genes have so far been found that cause MODY, although not all MODY patients have one of these genes. Because MODY runs in families, it is useful for studying diabetes genes and has given researchers useful information about how insulin is produced and regulated by the pancreas.

MDCK (Madin-Darby canine kidney) cells—Epithelial-like cell line established from normal kidney of dog, susceptible for many viral species.

Microcapsules—small particles that contain an active agent or core material surrounded by a coating or shell.

MMA (methyl methacrylate)—acrylic monomer, colorless liquid with a slight irritating odor.

NIT (NOD insulinoma tumor) cell line—cell line developed from pancreatic beta cells of a transgenic NOD mouse.

NVP (N-vinyl pyrrolidinone)—monomer produced from the reaction of acetylene with 2-Pyrrolidone. It serves as a reactive diluent in a variety of applications.

Nycodenz™ (Nycomed Pharma, Oslo, Norway)—Diatrizoic acid, a non-ionic X-ray contrast medium, used to make density gradients. A favorable property of Nycodenz solutions is that the osmolality and density can easily be varied over a broad range. An effective non-ionic, water-soluble contrast agent which is used in myelography, arthrography, nephroangiography, arteriography, and other radiographic procedures. Its low systemic toxicity is the combined result of low chemotoxicity and low osmolality.

Oral Glucose Tolerance Testing (OGTT)—A screening test for diabetes that involves testing an individual's plasma glucose level after he drinks a solution containing 75 grams of glucose. Currently, a person is diagnosed with diabetes if his plasma glucose level is 200 mg/dL or higher two hours after ingesting the glucose. Those with a plasma glucose level less than 200 mg/dL but greater than or equal to 140 mg/dL are diagnosed with a condition called impaired glucose tolerance. People with this condition have trouble metabolizing glucose, but the problem is not considered severe enough to classify them as diabetic. Individuals with impaired glucose tolerance are at a slightly elevated risk for developing high blood pressure, blood lipid disorders, and Type 2 diabetes.

Permselectivity—preferential permeation of certain ionic species through a membrane.

PoERV (porcine endogenous retrovirus)—An endogenous retrovirus exists as part of the DNA in all mammals and is passed down to offspring over successive generations.

postprandial—occurring after a meal

Proinsulin—a protein made by the pancreas beta cells which is cleaved into 3 units—C-peptide, alpha chain and beta chain. The alpha and beta chains are the functional units of insulin.

SGS (Static glucose stimulation)—static glucose challenge, evaluating the ability of the islets to secrete insulin in response to different glucose concentrations.

Streptozotocin—an antibiotic, C₈H₁₅N₃O₇, produced by an actinomycete (Streptomyces achromogenes) and active against tumors but damaging to insulin-producing cells and now also regarded as a carcinogen.

Theophylline—stimulates the release of catecholamines and reduces cerebral blood flow, thereby facilitating stronger metabolic responses to and a prompter perception of decreasing glucose levels.

Therapeutically effective amount—amount of a therapeutic agent produced by cells or tissue which, when administered to a subject in need thereof, is sufficient to effect treatment for a disease or disorder, or to effectively change the growth rate or alter the condition of an animal. The amount of encapsulated cells or tissue corresponding to a “therapeutically effective amount” will vary depending upon factors such as the disease condition and the severity thereof, the identity of the subject in need thereof, and the type of therapeutic agent delivered by the cells or tissue for the disease or disorder, but can nevertheless be readily determined by one of skill in the art.

Treating and Treatment—to alleviate a disease or disorder in a subject, such as a human, by the dosage of encapsulated cells or tissue to the subject in need of treatment via subcutaneous injection or implant, or directly into organs via either direct injection into the substance of the organ or injection through the vascular system of those organs and includes: (a) prophylactic treatment in a subject, particularly when the subject is found to be predisposed to having the disease or disorder but not yet diagnosed as having it; (b) inhibiting the disease or disorder; and/or (c) eliminating, in whole or in part, the disease or disorder; and/or (d) improving the subject's health and well-being.

Type 1 diabetes (also insulin-dependent diabetes, insulin-dependent diabetes mellitus)—a form of diabetes mellitus that usually develops during childhood or adolescence and is characterized by a severe deficiency in insulin secretion resulting from atrophy of the islets of Langerhans, and causing hyperglycemia and a marked tendency towards ketoacidosis.

Type 2 diabetes (also non-insulin-dependent diabetes, non-insulin-dependent diabetes mellitus)—a common form of diabetes mellitus that develops especially in adults and most often in obese individuals and that is characterized by hyperglycemia resulting from impaired insulin utilization coupled with the body's inability to compensate with increased insulin production.

Xenografts—A surgical graft of tissue from one species onto or into individuals of unlike species, genus or family. Also known as a heteroplastic graft.

### SUMMARY OF THE INVENTION

The present invention relates to methods of treating a disease or disorder by implanting encapsulated biological material into patients in need of treatment. Diabetes is of particular interest because a method is needed to prevent complications related to the lack of good glycemic control in insulin-requiring diabetics. The current complications of clinical islet transplantation and the significant risks and discomfort of continuous immunosuppression may be eliminated by applying the methods described herein to patients with insulin-requiring diabetes. In addition, encapsulated islet implants are expected to protect these insulin-requiring diabetic patients and prevent them from developing the complications from diabetes related to inadequate glycemic control in spite of exogenous insulin therapy.

Methods according to the present invention may provide therapeutic effects for a variety of diseases and disorders, in addition to diabetes, in which critical cell-based products lost by disease or disorder may be replaced through implantation of cells or tissue into the body.

An embodiment, is the use of human insulin-producing cells from the pancreas, or cells derived from human insulin-producing cells from the pancreas, that are encapsulated as cell clusters for implantation into the subcutaneous site of insulin-requiring patients. Treatment of disease via encapsulated biological materials requires that the encapsulated material be coated with a biocompatible coating, such that the immune system of the patient being treated does not destroy the material before a therapeutic effect can be realized.

Permselectivity of the coating is a factor in the effectiveness of such treatments, because this regulates the availability of nutrients to the cells or tissue, and plays a role in preventing rejection of the biological materials. Permselectivity of the coating affects the nutrition available to the encapsulated cell or tissue, as well as the function of the cell or tissue. Permselectivity can be controlled by varying the components of the biocompatible coating or by varying how the components are used to make the cell coating. Treatment via injection of encapsulated biological materials according to the present invention provides a stable and safe method of treatment. Size of the implant and the site of implantation, as well as replenishment and/or replacement of the encapsulated materials is also a consideration of the methods described herein. These methods provide a treatment that has a wide range of applications in the treatment of disease at various sites of implantation, while avoiding complications associated with other treatment methods.

The micro-bulk coatings described herein can be produced with different pore sizes that can be produced to limit access to the cells by proteins of widely varying molecular weights, including the exclusion of antibodies. This control allows for survival and maintained function of the encapsulated materials, while excluding components of the host immune system. The appropriate pore size of the micro-bulk coating may be determined by routine experimentation for each cell or tissue type and the disease or disorder to be treated. The micro-bulk coatings described herein provide a small encapsulated cell product with a minimal volume of the coating material, thus allowing the coated materials to be implanted into various sites of the body, including direct injection into the liver, spleen, muscle, or other organs, injection via vascular access to any organ, injection into the abdominal cavity, and implantation into a subcutaneous site.

An important factor for successful encapsulated cell therapy is that the permselective coating used to encapsulate the cells be inert in terms of causing inflammatory reactions in the host. Most previous encapsulating materials were not completely biocompatible. With some devices, not making a large scar is sufficient. However, when using the coating for permselective protection between the encapsulated cells and the host immune system, there cannot be any non-specific inflammatory reaction to the host's complement system or to macrophages. If this occurs, then the inflammatory and/or immune reaction is sufficient to release cytokines that readily cross the membrane and can cause the loss of the encapsulated cells. Most encapsulation technologies for islets, which have had difficulties in working appropriately, had non-specific inflammatory reactions due to biocompatibility reactions to the coating materials.

Problems such as chronic inflammation are significantly reduced due to the lack of host reaction to the biocompatible micro-bulk coatings used to encapsulate cells and tissues used in the methods described herein. The components used to produce the micro-bulk coating described herein have been shown to be completely biocompatible when injected into animals, such as, rodents, dogs, pigs, and primates.

We discovered that biocompatibility of hydrogels synthesized from highly acrylated PEG was exceptionally good, and much better than that shown with moderately acrylated PEG hydrogels. The highly acrylated PEGs were either obtained commercially, or home-made by acrylating corresponding PEGs. Hydrogels with highly acrylated PEGs were micro-bulk coated on the surface of alginate microbeads using an interfacial photopolymerization technology. This discovery also can be extended to other biomedical, biotechnological and pharmaceutical areas where biocompatibility of the devices or formulations is of concern.

Some PEG micro-bulk coatings described herein are biodegradable over time, thus allowing the body to safely break down the materials over the course of time and avoiding the need to retrieve the encapsulated materials, which is required by other treatments. Replacement of cells can be done whenever the previous dose of encapsulated materials has begun to lose function. Encapsulated islets may be expected to last two to five years or longer. In the case of subcutaneous injections, replacement of the encapsulated materials may simply be done via another percutaneous injection of new materials into the patient at a different site prior to loss of the previous dose. In the case of encapsulated islets, this replacement can be done prior to loss of function in the first dose of islets, without fear of low glucose values, because the encapsulated islets autoregulate themselves to prevent hypoglycemia. Different implant timing may have to be determined for treating diseases and disorders using cells or tissues that do not autoregulate the release of their product.

A factor in producing encapsulated cell products is the cell source. Cells may be primary cells, expanded cells, differentiated cells, cell lines, or genetically engineered cells. In the case of human islets, primary islets may be isolated from cadaver-donated pancreases; however, the number of human pancreata available for isolating islets is very limited. Alternative cell sources may be used to provide cells for encapsulation and injection.

One alternative source of cells, particularly insulin-producing cells, is embryonic stem cells. Human embryonic stem cells come from the very early fetus. They are only available when grown from frozen, fertilized human eggs collected from couples that have successfully undergone in vitro fertilization and no longer want to keep these fertilized eggs for future children. Embryonic stem cells have the ability to grow indefinitely, potentially avoiding the need for the mass of tissues required for transplantation. There are a series of steps required to differentiate these embryonic stem cells into insulin producing cells with clinical relevancy. A few studies have shown both mouse and human embryonic stem cells can produce insulin when treated under tissue culture with a variety of factors. Insulin-producing cells developed from embryonic stem cells may be an acceptable cell source for transplantation, and encapsulated cell or tissue implantation.

### Cell Sourcing

Additional cell sources, organ specific progenitor cells from the brain, liver, and the intestine, have been shown to produce insulin. In order to produce insulin, each of these organ specific progenitor cells has undergone tissue culture treatments with a variety of growth and differentiation factors. Additional organ specific progenitor cells from many other organs such as bone marrow, kidney, spleen, muscle, bone, cartilage, blood vessels, and other endocrine organs may also be useful in providing insulin producing cells.

Pancreatic progenitor cells may be used according to the methods. The pancreas seems to have organ specific stem cells that can produce the three pancreatic cell types in the body under normal and repair conditions. It is believed the islet cells bud off from the duct cells to form the discrete islets. The insulin producing beta cells, as well as the other hormone producing cells, may form directly from differentiating duct cells or may form from pancreatic progenitor cells located amongst the duct cells. These pancreatic progenitor cells may be used to provide insulin-producing cells for encapsulation and implantation according to the methods described herein.

There has been a great deal of research on genetically inserting genes into non-insulin producing cells to make them produce insulin. Genetically engineered cells capable of insulin production may also be used for encapsulation and implantation according to the methods described herein.

The use of pig cells has commonly been considered as a source of islet cells for implantation in patients with diabetes. Over 90 million pigs are raised per year for meat production in the USA alone. Therefore, the number of islets to treat the millions of patients with insulin-requiring diabetes is readily available through large scale processing of adult pig pancreata into purified pig islets for encapsulation. One consideration limiting this choice is the recognition that pigs harbor an endogenous retrovirus (PoERV). There have been efforts to eliminate PoERV from strains of pigs. Virus-free pig xenograft islets may be readily encapsulated and available as a preferred cell source for the treatment of human diabetes.

Alternative xenograft sources for human implantation may be obtained from primary cells of species other than pigs. These other species could be agriculturally relevant animals such as beef, sheep, and even fish. With the ability to expand and differentiate insulin producing cells from pancreatic sources or other stem or progenitor cells, one can envision using insulin-producing cells from many other xenogeneic sources such as primates, rodents, rabbits, fish, marsupials, ungulates and others.

### Disease Treatment

Diabetes and other diseases in which a local or circulating factor is deficient or absent can be treated according to the methods described herein. Encapsulated cell therapy may be applied in the treatment of neurologic, cardiovascular, hepatic, endocrine, skin, hematopoietic, and immune disorders and diseases. Neurologic diseases and injuries, such as Parkinson's disease, Alzheimer's disease, Huntington's disease, multiple sclerosis, blindness, spinal cord injury, peripheral nerve injury, pain and addiction may be treated by encapsulating cells that are capable of releasing local and/or circulating factors needed to treat these problems. Cardiovascular tissue, such as the coronary artery, as well as angiogenic growth factor releasing cells used for restoring vascular supply to ischemic cardiac muscle, valves and small vessels may be treated. Acute liver failure, chronic live failure, and genetic diseases affecting the liver may be treated. Endocrine disorders and diseases, such as diabetes, obesity, stress and adrenal, parathyroid, testicular and ovarian diseases may be treated. Skin problems, such as chronic ulcers, and diseases of the dermal and hair stem cells can be treated. Hematopoietic factors such as Factor VIII and erythropoietin may be regulated or controlled by administering cells capable of stimulating a hematopoietic response in a patient. Encapsulated biological materials may also be useful in the production of bone marrow stem cells. Encapsulated materials, such as, antigens from primary cells or genetically engineered cells, may be useful in producing immune tolerance or preventing autoimmune disease. In addition, these materials may be used in vaccines.

### Micro-Bulk Coating Components

Components of the coatings may be altered depending on the specific cell type and permselectivity desired. Various polymerizable monomers or macromers, photoinitiating dyes, cocatalysts, and accelerants may be used to produce micro-bulk coated cells and tissues.

### Monomers or Macromers

Monomers or macromers are used as the building blocks to polymerize biocompatible coatings for use in methods disclosed herein. The monomers are small polymers, which are susceptible to polymerization into the larger polymer membranes of this invention. Polymerization is enabled because the macromers contain carbon-carbon double bond moieties, such as, acrylate, methacrylate, ethacrylate, 2-phenyl acrylate, 2-chloro acrylate, 2-bromo acrylate, itaconate, acrylamide, methacrylamide, and styrene groups. The monomers or macromers are non-toxic to biological material before and after polymerization.

Examples of monomers are methyl methacrylate (MMA) and 2-hydroxyethyl methacrylate (HEMA). Examples of macromers are ethylenically unsaturated derivatives of poly(ethylene oxide) (PEO), poly(ethylene glycol) (PEG), poly(vinyl alcohol) (PVA), poly(vinylpyrrolidone) (PVP), poly(thyloxazoline) (PEOX), poly(amino acids), polysaccharides such as alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives and carrageenan, and proteins such as gelatin, collagen and albumin. These macromers can vary in molecular weight and number of branches, depending on the use. For purposes of encapsulating cells and tissue in a manner that has minimum tissue response, the preferred starting macromer is PEG—triacrylate with MW 1.1K. The molecular weight designation is an average molecular weight of the mixed length polymer.

### Photoinitiating Dyes

The photoinitiating dyes capture light energy and initiate polymerization of the macromers and monomers. Any dye can be used which absorbs light having frequency between 320 nm and 900 nm, can form free radicals, is at least partially water soluble, and is non-toxic to the biological material at the concentration used for polymerization. Examples of suitable dyes are ethyl eosin, eosin Y, fluorescein, 2,2-dimethoxy, 2-phenylacetophenone, 2-methoxy, 2-phenylacetophenono, camphorquinone, rose bengal, methylene blue, erythrosin, phloxine, thionine, riboflavin and methylene green. To enhance the dye-cell surface binding, the dyes used here are conjugated to polymers that have strong interactions with cell surfaces, such as polycationic polymers, polymers with multiple phenylboronic acid groups attached. Examples of polycationic polymers include PAMAM dendrimer, linear, branched or dendritic poly (ethyleneimine) (PEI), polyvinylamine, polyallylamine, polylysine, chitosan, and polyhistidine. The preferred initiator dye is the carboxyeosin conjugated to PAMAM Dendrimer Generation 4.

### Cocatalyst or Radical Generator

The cocatalyst is a nitrogen-based compound capable of stimulating the free radical reaction. Primary, secondary, tertiary or quaternary amines are suitable cocatalysts, as are any nitrogen atom containing electron-rich molecules. Cocatalysts include, but are not limited to, triethanolamine, triethylamine, ethanolamine, N-methyl diethanolamine, N,N-dimethyl benzylamine, dibenzyl amino, N-benzyl ethanolamine, N-isopropyl benzylamine, tetramethyl ethylenediamine, potassium persulfate, tetramethyl ethylenediamine, lysine, omithine, histidine and arginine. A preferred cocatalyst is triethanolamine.

### Accelerator or Co-Monomer

The accelerator, which is optionally included in the polymerization mixture, is a small molecule containing an allyl, vinyl, or acrylate group, and is capable of speeding up the free radical reaction. Incorporating a sulfonic acid group to the accelerant also can improve the biocompatibility of the final product. Accelerators include, but are not limited to, N-vinyl pyrrolidinone, 2-vinyl pyridine, 1-vinyl imidazole, 9-vinyl carbazone, 9-vinyl carbozol, acrylic acid, 2-allyl-2-methyl-1,3-cyclopentane dione, 2-hydroxyethyl acrylate, 2-acrylamido-2-methyl-1-propanesulfonic acid, vinylsulfonic acid, 4-styrenesulfonic acid, 3-sulfopropyl acrylate, 3-sulfopropyl methacrylate, n-vinylcarpolactam, and n-vinyl maleimide sulfonate (from SurModics), with 2-acrylamido-2-methyl-1-propanesulfonic acid plus N-vinyl pyrrolidinone being the preferred combination of accelerators.

### Viscosity Enhancer

To generate micro-bulk coating without long tails on cell aggregates, the viscosity of the macromer solution may be optimized. This may be accomplished by viscosity enhancers which are added into the macromer solution. Preferred viscosity enhancers are PEG—triol with MW 3.5 kD and 4 kD PEG-diol.

### Density Adjusting Agent

To generate micro-bulk coating without long tails on cell aggregates, the density of the macromer solution may be optimized. This may be accomplished by adding density adjusting agents into the macromer solution. Preferred density adjusting agents are Nycodenz™ and Ficoll™.

### Radiation Wavelength

The radiation used to initiate the polymerization is either longwave UV or visible light, with a wavelength in the range of 320-900 nm, the range of 350-700 nm, or the range of 365-550 nm, is used. This light can be provided by any appropriate source able to generate the desired radiation, such as a LED, mercury lamp, longwave UV lamp, He—Ne laser, or an argon ion laser or an appropriately filtered xenon light source.

### BRIEF DESCRIPTION OF THE DRAWING

FIG. 1A is a photomicrograph of Alginate capsules.

FIG. 1B is a photomicrograph of encapsulated islets.

FIG. 1C is a photomicrograph of PEG Conformal Coated Islets.

FIG. 2A is a photomicrograph of Empty Alginate Capsules in Nu/Nu Mice—Histology without fragmentation.

FIG. 2B is a photomicrograph of Empty Alginate Capsules in C57Bl6 mice—Histology without fragmentation.

FIG. 2C is a photomicrograph of IP Empty PEG-Diacrylate Capsules in Nu/Nu Mice at 2 weeks—In vivo.

FIG. 2D is a photomicrograph of IP Empty PEG-Diacrylate Capsules in Nu/Nu Mice at 2 weeks—Ex vivo.

FIG. 2E is a photomicrograph of cell histology.

FIG. 2F is a photomicrograph of cell histology.

FIG. 2G is a photomicrograph of IP Empty PEG-Diacrylate Capsules in C57Bl6 Mice at 2 weeks—In vivo.

FIG. 2H is a photomicrograph of IP Empty PEG-Diacrylate Capsules in C57Bl6 Mice at 2 weeks—Ex vivo.

FIG. 2I is a photomicrograph of cell histology.

FIG. 2J is a photomicrograph of cell histology.

FIG. 3 is a photograph of an Unconstrained Compression Testing to Measure PEG Capsule Strength.

FIG. 4 shows the Test Results of Elasticity of PEG-Diacrylate Capsules.

FIG. 5 shows Capsule Size Distributions.

FIG. 6A is a photomicrograph of PEG 5% after transplant from nude mice.

FIG. 6B is a photomicrograph of PEG 5% after transplant from BL6 mice.

FIG. 6C is a photomicrograph of 5% PEG-DA Histology—Nu/Nu Mouse Implants.

FIG. 6D is a photomicrograph of 5% PEG-DA Histology—C57Bl6 Mouse Implants.

FIG. 6E is a photomicrograph of PEG 7.5% after transplant from nude mice.

FIG. 6F is a photomicrograph of PEG 7.5% after transplant from BL6 mice.

FIG. 7 is a photomicrograph of PEG-Acrylates under testing.

FIG. 8 shows the elasticity of different PEG-Acrylates.

FIG. 9A is a photomicrograph of 5% 1 kDa PEG Diacrylate with 150 sec light exposure (dead islets).

FIG. 9B is a photomicrograph of 7.5% 1 kDa PEG Diacrylate with 150 sec light exposure (dead islets).

FIG. 9C is a photomicrograph of 5% 10 kDa PEG-tetra-Acrylate with 20 sec light exposure (viable islets).

FIG. 9D is a photomicrograph of 10% 10 kDa PEG-tetra-Acrylate with 20 sec light exposure (viable islets).

FIG. 10A is a photomicrograph of Nu/Nu mice Implants empty capsules.

FIG. 10B is a photomicrograph of Nu/Nu mice Implants empty capsules.

FIG. 10C is a photomicrograph of C57Bl6 mice Implants empty capsules.

FIG. 10D is a photomicrograph of C57Bl6 mice Implants empty capsules.

FIG. 10E is a photomicrograph of Nu/Nu mice implants+human islets.

FIG. 10F is a photomicrograph of C57Bl6 mice implants+human islets.

FIGS. 11A, B & C are Black & White photomicrographs of micro-bulk PEG Capsules (Bar=1000 microns) First micro-bulk PEG capsules containing human islets produced at 1-2 mm size. (dark spots).

FIG. 11D shows the Viability test (EB/FDA stain) of micro-bulk PEG encapsulated human islets. (Green=Viable).

FIG. 11E shows the Functional Glucose Stimulated Insulin Release (GSIR) test results on these first, large micro-bulk PEG capsules. 1^{st} Stim Index=12 mM/3 mM, 2^{nd} Stim Index=25 mM/3 mM, 3^{rd} Stim Index=25 mM+IBMX/3 mM).

FIGS. 11F & G is a photomicrograph of Starting Size of micro-bulk PEG capsules with human islets >1000 microns (Bar=1000 microns).

FIGS. 11H, I & J is a photomicrograph of First Size Reduction Step to the 1000 to the 500 micron range with human islets. (Bar=1000 microns).

FIGS. 11K, L & M is a photomicrograph of Second Size Reduction Step to the <500 micron range with human islets. (Bar equals 400 micron).

### DETAILED DESCRIPTION OF THE INVENTION

This invention provides novel methods for the formation of biocompatible membranes around biological materials using photopolymerization of water-soluble molecules. The membranes can be used as a covering to encapsulate biological materials or biomedical devices, as a “glue” to cause more than one biological substance to adhere together, or as carriers for biologically active species.

Several methods for forming these membranes are provided. Each of these methods utilizes a polymerization system containing water-soluble macromers, species, which are at once polymers and macromolecules capable of further polymerization. The macromers are polymerized using a photoinitiator (such as a dye), optionally a cocatalyst, optionally an accelerator, and radiation in the form of visible or long wavelength UV light. The reaction occurs either by suspension polymerization or by interfacial polymerization. The polymer membrane can be formed directly on the surface of the biological material, or it can be formed on material, which is already encapsulated.

Ultrathin membranes can be formed-by the methods described herein. These ultrathin membranes allow for optimal diffusion of nutrient and bioregulator molecules across the membrane, and great flexibility in the shape of the membrane. Such thin membranes produce encapsulated material with optimal economy of volume. Biological material thus coated can be packed into a relatively small space without interference from bulky membranes.

The thickness and pore size of membranes formed can be varied. This variability allows for “perm-selectivity”—membranes can be adjusted to the desired degree of porosity, allowing only preferred molecules to permeate the membrane, while acting as a barrier against larger undesired molecules. Thus, the membranes are immunoprotective in that they prevent the transfer of antibodies or cells of the immune system.

When the encapsulated biological material is cellular in nature, the absence of small monomers in the polymerization solution prevents the diffusion of toxic molecules into the cell. In this manner the present invention provides a polymerization system which is more biocompatible than any available in the prior art.

Additionally, the polymerization method utilizes short bursts of visible or long wavelength UV light, which is nontoxic to biological material. Bioincompatible polymerization initiators employed in the prior art are also eliminated.

According to the present invention, membrane formation occurs under physiological conditions. Thus, no damage is done to the enclosed biological material due to harsh pH, temperature, or ionic conditions.

Because the membrane adheres to the biological material, the membrane can be used as an adhesive to fasten more than one biological substance together. The macromers are polymerized in the presence of these substances which are in close proximity. The membrane forms in the interstices, effectively gluing the substances together.

Additionally, utilizing the tendency of the membrane to adhere to biological material, a membrane can be formed around or on a biologically active substance to act as a carrier for that substance.

In one embodiment, the invention is directed to a composition for cellular therapy, which includes a plurality of encapsulating devices comprising a micro-bulk coating including a polyethylene glycol (PEG) coating, said PEG having a molecular weight between about 900 and about 20,000 Daltons; and a plurality of cells encapsulated in the encapsulating devices, wherein said composition has a cell density of at least about 100,000 cells/ml and a sulfonated comonomer, and wherein the micro-bulk coating comprises salt, MOPS (3-(N-morpholino)propanesulfonic) acid, co-monomer, a diol containing compound, an x-ray contrast agent and a photo-initiator.

In one embodiment, the encapsulating devices are microcapsules. In a one embodiment, the microcapsules are micro-bulk coated cell aggregates.

In one embodiment, the cell aggregates are pancreatic islets with a cell density which is at least about 100,000 cells/ml.

In one embodiment, the cell is neurologic, cardiovascular, hepatic, endocrine, skin, hematopoietic, immune, neurosecretory, metabolic, systemic, or genetic. In one embodiment, the cell is autologous, allogeneic, xenogeneic or genetically-modified. In one embodiment, the cell is an insulin producing cell.

In one embodiment, the PEG is a diacrylate of PEG with a molecular weight in the range of 2 kD to 16 kD. In one embodiment, the PEG is a triacrylate of PEG with a molecular weight in the range of 3 kD to 16 kD. In one embodiment, the PEG is a tetra-acrylate of PEG with a molecular weight in the range of 4 kD to 20 kD.

In one embodiment, the PEG is a combination of a diacrylate of PEG with a molecular weight in the range of 2 kD to 16 kD, a triacrylate of PEG with a molecular weight in the range of 3 kD to 16 kD and a tetra-acrylate of PEG with a molecular weight in the range of 4 kD to 20 kD.

In one embodiment, the co-monomer is AMPS (2-Acrylamido-2-methylpropane sulfonic acid), ammonium AMPS, 2-methyl-2-((1-oxo-2-propenyl)amino)-monoammomium salt, nVP (N-Vinylpyrrolidone), polyvidone, polyvinylpolypyrrolidone or similar types of co-polymers.

In one embodiment, the x-ray contrast agent is nycodenz, iohexol, omnipaque or similar low-osmolality agents.

In one embodiment, the salt has the formula of XCl₂(H₂O)ₐ; where X=calcium, magnesium, barium or strontium; and a=0, 1, 2, 4 or 6.

In one embodiment, the photo-initiator is eosin Y, tetrabromo derivative of fluorescein, methylated eosin Y, ethylated eosin Y, eosin yellowish, bromofluoresceic acid, acid red 87, bromoeosine, eosin B, dibromo dinitro derivative of fluorescein, or similar compounds.

In one embodiment, the diol containing compound is PEG-diol, beta propylene glycol, propylene-1,3,diol, bisphenol A, 1,4-butanediol or similar compounds.

In one embodiment, the micro-bulk capsule envelopes the cell aggregate.

In another embodiment, the invention is directed to a therapeutically effective composition which includes a plurality of encapsulating devices having an average diameter of less than 400 micron, where the encapsulating devices include encapsulated cells in an encapsulation material, and the composition comprises at least about 500,000 cells/ml.

In one embodiment, the average diameter of the encapsulating device is less than 300 micron. In one embodiment, the average diameter of the encapsulating device is less than 200 micron. In one embodiment, the average diameter of the encapsulating device is less than 100 micron. And in one embodiment, the average diameter of the encapsulating device is less than 50 micron.

In on embodiment, the invention is directed to a therapeutically effective composition including a plurality of encapsulating devices having an average diameter of less than 400 micron, where the encapsulating devices include encapsulated cells in an encapsulation material, and the composition has a ratio of volume of encapsulating device to volume of cells of less than about 20:1.

In one embodiment, the composition has a ratio of volume of encapsulating device to volume of cells of less than about 10:1. In one embodiment, the composition has a ratio of volume of encapsulating device to volume of cells of less than about 2:1.

In another embodiment, the invention is directed to using a therapeutic composition as described herein in a method which includes the step of implanting the composition into an implantation site in an animal in need of treatment for a disease or disorder.

In one embodiment, the invention is directed to a method of using the therapeutic composition which includes encapsulating devices with a polyethylene glycol (PEG) coating having a molecular weight between 900 and 20,000 Daltons, where the composition has a cell density of at least about 100,000 cells/ml in a method which includes the step of implanting the composition into an implantation site in an animal in need of treatment for a disease or disorder.

In one embodiment, the implanting is an injection.

In one embodiments, the disease or disorder is neurologic, cardiovascular, hepatic, endocrine, skin, hematopoietic, immune, neurosecretory, metabolic, systemic, or genetic.

In one embodiment, the disease is an endocrine disease which is diabetes.

In one embodiment, the animal is from an Order of Subclass Theria which is Artiodactyla, Carnivora, Cetacea, Perissodactyla, Primate, Proboscides, or Lagomorpha. In one embodiment, the animal is a Human.

In one embodiment, the implantation site is subcutaneous, intramuscular, intraorgan, arterial/venous vascularity of an organ, cerebro-spinal fluid, or lymphatic fluid. In one embodiment, the implantation site is subcutaneous.

In one embodiment, the method includes implanting encapsulated islets in a subcutaneous implantation site.

In one embodiment, the method of implanting the composition into an implantation site in an animal in need of treatment for a disease or disorder also includes the step of administering an immunosuppressant or anti-inflammatory agent.

In one embodiment, the immunosuppressant or anti-inflammatory agent is administered for less than 6 months. In one embodiment, the immunosuppressant or anti-inflammatory agent is administered for less than 1 month.

In another one embodiment, the invention is directed to using a therapeutic composition which includes a plurality of encapsulating devices having an average diameter of less than 400 micron, where the encapsulating devices include encapsulated cells in an encapsulation material and the composition has at least about 500,000 cells/ml, in a method which includes the step of implanting the composition into an implantation site in an animal in need of treatment for a disease or disorder.

In one embodiment, the implantation is an injection.

In one embodiment, the animal is from an Order of Subclass Theria which is Artiodactyla, Carnivora, Cetacea, Perissodactyla, Primate, Proboscides, or Lagomorpha. In one embodiment, the animal is a Human.

In one embodiment, the implantation site is subcutaneous, intramuscular, intraorgan, arterial/venous vascularity of an organ, cerebro-spinal fluid, or lymphatic fluid. In one embodiment, the implantation site is subcutaneous.

In one embodiment, the method includes implanting encapsulated islets in a subcutaneous implantation site. In one embodiment, the method of implanting the composition into an implantation site in an animal in need of treatment for a disease or disorder also includes the step of administering an immunosuppressant or anti-inflammatory agent.

In one embodiment, the immunosuppressant or anti-inflammatory agent is administered for less than 6 months. In one embodiment, the immunosuppressant or anti-inflammatory agent is administered for less than 1 month.

In one embodiment, the encapsulated biological material is a PEG micro-bulk coated islet allograft. In one embodiment, the biological material is an organ, tissue or cell. In one embodiment, the tissue is a cluster of insulin producing cells. In one embodiment, the cell is an insulin producing cell.

In one embodiment, the photoinitiator is carboxyeosin, ethyl eosin, eosin Y, fluorescein, 2,2-dimethoxy, 2-phenylacetophenone, 2-methoxy, 2-phenylacetophenono, camphorquinone, rose bengal, methylene blue, erythrosin, phloxine, thoionine, riboflavin or methylene green.

In one embodiment, the photoactive polymer solution includes a polymerizable high density ethylenically unsaturated PEG and a sulfonated comonomer.

In a one embodiment, the polymerizable high density ethylenically unsaturated PEG is a high density acrylated PEG. In a one embodiment, the polymerizable high density acrylated PEG has a molecular weight of 1.1 kD.

In one embodiment, the sulfonated comonomer is 2-acrylamido-2-methyl-1-propanesulfonic acid, vinylsulfonic acid, 4-styrenesulfonic acid, 3-sulfopropyl acrylate, 3-sulfopropyl methacrylate, or n-vinyl maleimide sulfonate. In a one embodiment, the sulfonated comonomer is 2-acrylamido-2-methyl-1-propanesulfonic acid.

In one embodiment, the photoactive polymer solution also includes a cocatalyst which is triethanolamine, triethylamine, ethanolamine, N-methyl diethanolamine, N,N-dimethyl benzylamine, dibenzyl amino, N-benzyl ethanolamine, N-isopropyl benzylamine, tetramethyl ethylenediamine, potassium persulfate, tetramethyl ethylenediamine, lysine, omithine, histidine or arginine.

In one embodiment, the cocatalyst is triethanolamine.

In one embodiment, the photoactive polymer solution also includes an accelerator which is N-vinyl pyrrolidinone, 2-vinyl pyridine, 1-vinyl imidazole, 9-vinyl carbazone, 9-vinyl carbozol, acrylic acid, n-vinylcarpolactam, 2-allyl-2-methyl-1,3-cyclopentane dione, or 2-hydroxyethyl acrylate.

In one embodiment, the accelerator is N-vinyl pyrrolidinone.

In one embodiment, the photoactive polymer solution also includes a viscosity enhancer which is selected from the group including natural and synthetic polymers. In a one embodiment, the viscosity enhancer is 3.5 kD PEG-triol or 4 kD PEG-diol.

In one embodiment, the biological material for the encapsulation method is neurologic, cardiovascular, hepatic, endocrine, skin, hematopoietic, immune, neurosecretory, metabolic, systemic, or genetic. In one embodiment, the biological material is from an animal of Subclass Theria of Class Mammalia. In a one embodiment, the animal is from an Order of Subclass Theria which is Artiodactyla, Carnivora, Cetacea, Perissodactyla, Primate, Proboscides, or Lagomorpha. In one embodiment, the animal is a Human.

In another embodiment, the invention is directed to a composition for encapsulating biological material which includes a polymerizable high density ethylenically unsaturated PEG having a molecular weight between 900 and 20,000 Daltons, and a sulfonated comonomer.

In one embodiment, the composition for encapsulating biological material has the quality of permselectivity. In one embodiment, the permselectivity can be engineered by manipulating the composition.

In one embodiment, the composition for encapsulating biological material further is biodegradable. In one embodiment, the composition is biodegradable in a mammal. In one embodiment, the composition is biodegradable in a sub-human primate. In one embodiment, the composition is biodegradable in a human.

Further aspects, features and advantages of this invention will become apparent from the detailed description of the one embodiments which follow.

One embodiment, is related to compositions and methods of treating one or more diseases or disorders, such as neurologic (e.g., Parkinson's disease, Alzheimer's disease, Huntington's disease, Multiple Sclerosis, blindness, peripheral nerve injury, spinal cord injury, pain and addiction), cardiovascular (e.g., coronary artery, angiogenesis grafts, valves and small vessels), hepatic (e.g., acute liver failure, chronic liver failure, and genetic diseases effecting the liver), endocrine (e.g., diabetes, obesity, stress and adrenal, parathyroid, testicular and ovarian diseases), skin (e.g., chronic ulcers and diseases of the dermal and hair stem cells), hematopoietic (e.g., Factor VIII and erythropoietin), or immune (e.g., immune intolerance or auto-immune disease), in a subject in need of treatment comprising: providing cells or tissue, such as pancreatic islets, hepatic tissue, endocrine tissues, skin cells, hematopoietic cells, bone marrow stem cells, renal tissues, muscle cells, neural cells, stem cells, embryonic stem cells, or organ specific progenitor cells, or genetically engineered cells to produce specific factors, or cells or tissue derived from such; enclosing said cells or tissue within at least one encapsulating material, such as a hydrogel, made of physically or chemically crosslinkable polymers, including polysaccharides such as alginate, agarose, chitosan, poly(amino acids), hyaluronic acid, chondroitin sulfate, dextran, dextran sulfate, heparin, heparin sulfate, heparan sulfate, gellan gum, xanthan gum, guar gum, water soluble cellulose derivatives, carrageenan, or proteins, such as gelatin, collagen, albumin, or water soluble synthetic polymers with ethylenically unsaturated groups or their derivatives, such as poly(methyl methacrylate) (PMMA), or poly(2-hydroxyethyl methacrylate) (PHEMA), poly(ethylene glycol) (PEG), poly(ethylene oxide) (PEO), poly(vinyl alcohol) (PVA), poly(vinylpyrrolidone) (PVP), poly(thyloxazoline) (PEOX); or a combination of the above, such as alginate mixed with PEG, or more hydrophobic or water insoluble polymers, such as poly(glycolic acid) (PGA), poly(lactic acid) (PLA), or their copolymers (PLA-GA), or polytetrafluoroethylene (PTFE) and administering a therapeutically effective amount of said encapsulated cells or tissue to the subject in need of treatment via subcutaneous injection or implant, or directly into organs via either direct injection into the substance of the organ or injection through the vascular system of those organs.

Organs maybe selected from, but not limited to, liver, spleen, kidney, lung, heart, brain, spinal cord, muscle, and bone marrow.

The subject in need of treatment may be selected from, but not limited to, mammals, such as humans, sub-human primates, cows, sheep, horses, swine, dogs, cats, and rabbits as well as other animals such as chickens, turkeys, or fish.

In a further embodiment, the encapsulated cell or tissue may be administered to a subject in need of treatment in combination with an immunosuppressant and/or an anti-inflammatory agent. The immunosuppressant may be selected from, but not limited to cyclosporine, sirolimus, rapamycin, or tacrolimus. The anti-inflammatory agent may be selected from, but not limited to, aspirin, ibuprofen, steroids, and non-steroidal anti-inflammatory agents.

The immunosuppressant and/or an anti-inflammatory agent is administered for six months following implantation or injection of the encapsulated cells or tissue. The immunosuppressant and/or an anti-inflammatory agent is administered for one month following implantation or injection of the encapsulated cells or tissue

In a embodiment, encapsulated islets are implanted or injected subcutaneously or into liver or spleen. In one aspect, micro-bulk coated islets are administered subcutaneously.

In some embodiments, the concentration of ingredients and composition of encapsulating solution may vary. Concentration ranges are as follows.

For Buffer solution a concentration is 1 to 200 mM, 5 to 100 mM, and 10 to 50 mM.

For CaCl₂ a concentration is 0.1 to 40 mM, 0.5 to 20 mM, and 1 to 5 mM. For Manitol a concentration is 10 mM to 6M, yet more is 50 mM to 3M, yet more is 100 mM to 1M, and yet more is 200 to 300 mM.

For pH of CaCl₂/Manitol solution a value is 6 to 8, 6.4 to 7.6, and 6.6 to 7.4.

For DEN-EY a concentration is 0.005 to 8 mg/ml, 0.01 to 4 mg/ml, and 0.05 to 2 mg/ml.

For DEN-EY conjunction level a level is 0.15 to 68, 1 to 34, and 1.5 to 15.

For pH of macromer solution a value is 6.5 to 9.5, 7 to 9, and 7.5 to 8.5.

For PEG TA a concentration is 0.1 to 100%, 0.2 to 50%, and 1 to 25%.

For PEG TA a density is 0.05 to 20 K, 0.1 to 10 K, 0.5 to 5 K, and 0.8 to 2.5 K.

For PEG-triol a concentration is 0.1 to 100%, 1 to 75%, and 2 to 50%.

For PEG-triol a density is 0.15 to 70 K, 0.3 to 35 K, 1.5 to 15 K, and 2.3 to 7.5 K.

For PEG-diol a concentration is 0.1 to 100% 1 to 75%, and 2 to 50%.

For PEG-diol a density is 0.2 to 80 K, 0.5 to 40 K, 1 to 20 K, and 2 to 10 K.

For TEoA a concentration is 5 mM to 2 M, 10 mM to 1M, 50 to 500 mM, and 75 to 125 mM.

For AMPS a concentration is 2 to 640 mg/ml, 5 to 300 mg/ml, and 10 to 150 mg/ml.

For NVP a concentration is 0.01 to 40 μl/ml, 0.1 to 20 μl/ml, and 0.5 to 10 μl/ml.

For Nycodenz a concentration is 0.1 to 100%, 1 to 50%, and 5 to 25%.

For the Laser a strength is 10 mW/cm² to 4 W/cm², 25 mW/cm² to 2 W/cm², and 75 mW/cm² to 1 W/cm².

For the light source a time is 3 seconds to 20 minutes, 6 seconds to 10 minutes, and 12 seconds to 3 minutes.

In an embodiment, the encapsulating material comprises a hydrogel that forms a sphere around at least one cell or tissue.

In one embodiment, a cell or tissue may be encapsulated in a biocompatible alginate microcapsule, wherein the alginate is made biocompatible by coating the alginate in a biocompatible material, such as PEG or hyaluronic acid, purifying the alginate and/or removing the poly-lysine and replacing it with PEG.

The disease to be treated is diabetes, the cells or tissue comprise insulin producing cells or tissue, or cells or tissue derived from pancreatic cells or tissue, or cells derived from progenitor or stem cells that are converted into insulin producing cells, and the encapsulated cells or tissue are administered to the subject in need of treatment via subcutaneous or liver injection or implant.

According to an embodiment the microcapsules of encapsulated insulin-producing cells or tissue may have an average diameter of 10 micron to 1000 micron, 100 micron to 600 micron, 150 micron to 500 micron, and 200 micron to 300 micron.

In another embodiment, the invention relates to an insulin-producing cell or tissue encapsulated in microcapsules having a concentration of at least 2,000 IEQ (islet equivalents)/ml, at least 9,000 IEQ/ml, and at least 200,000 IEQ/ml.

In another embodiment, the volume of insulin-producing cells or tissue encapsulated in microcapsules administered per kilogram body mass of a subject may be 0.001 ml to 10 ml, 0.01 ml to 7 ml, 0.05 ml to 2 ml.

In one embodiment, the ratio of microcapsule volume to insulin producing cell or tissue volume is less than 300 to 1, less than 100 to 1, less than 50 to 1, and less than 20 to 1.

In one embodiment, micro-bulk coated insulin-producing cells or tissue may have an average membrane thickness of 1 to 400 micron, 10 to 200 micron, and 10 to 100 micron. In one embodiment the invention relates to a micro-bulk coated insulin-producing cell or tissue having a concentration of at least 10,000 IEQ/ml, at least 70,000 IEQ/ml, at least 125,000 IEQ/ml, and at least 200,000 IEQ/ml.

In one embodiment, the volume of the micro-bulk coated insulin producing cell or tissue administered per kilogram body mass of a subject may be 0.01 to 7 ml, 0.01 to 2 ml, and 0.04 to 0.5 ml.

In one embodiment, the ratio of micro-bulk coating volume to insulin-producing cell or tissue volume is less than 13 to 1, less than 8 to 1, less than 5 to 1, and less than 2.5 to 1.

In one embodiment, the microcapsules of encapsulated cells or tissue may have an average diameter of 10 micron to 1000 micron, 100 micron to 600 micron, 150 micron to 500 micron, and 200 micron to 300 micron.

In one embodiment, the ratio of microcapsule volume to insulin producing cell or tissue volume is less than 300 to 1, less than 100 to 1, less than 50 to 1, and less than 20 to 1.

In one embodiment, micro-bulk coated cells or tissue may have an average membrane thickness of 1 to 400 micron, 10 to 200 micron, and 10 to 100 micron.

In one embodiment, the ratio of micro-bulk coating volume to cell or tissue volume is less than 13 to 1, less than 8 to 1, less than 5 to 1, and less than 2.5 to 1.

In one embodiment, relates encapsulated cells or tissue where the cell density is at least about 100,000 cells/ml. The encapsulated cell is micro-bulk coated. The cell is micro-bulk coated with an encapsulating material comprising acrylated PEG.

In one embodiment, a method of treating diabetes in a subject comprising administering encapsulated islets where the cell density is at least about 6,000,000 cells/ml, where the curative dose is less than about 2 ml per kilogram body mass of the subject.

In one embodiment, related to agricultural animals or pets, such as cows, sheep, horses, swine, chickens, turkeys, rabbits, fish, or dogs and cats; to change the growth rate, or alter the condition of the animal (e.g., increase meat or dairy production), or protect them from or treat them for different diseases.

In one embodiment, a method of providing cells or tissue to an agriculturally relevant animal comprises: a) providing a cell or tissue; b) enclosing said cell or tissue within at least one encapsulating material, such as a hydrogel, made of physically or chemically crosslinkable polymers, including polysaccharides such as alginate, agarose, chitosan, poly(amino acids), hyaluronic acid, chondroitin sulfate, dextran, dextran sulfate, heparin, heparin sulfate, heparan sulfate, gellan gum, xanthan gum, guar gum, water soluble cellulose derivatives, carrageenan, or proteins, such as gelatin, collagen, albumin, or water soluble synthetic polymers or their derivatives, such as methyl methacrylate (MMA), or 2-hydroxyethyl methacrylate (HEMA), polyethylene glycol (PEG), poly(ethylene oxide) (PEO), poly(vinyl alcohol) (PVA), poly(vinylpyrrolidone) (PVP), poly(thyloxazoline) (PEOX); or a combination of the above, such as alginate mixed with PEG, or more hydrophobic or water insoluble polymers, such as poly(glycolic acid) (PGA), poly(lactic acid) (PLA), or their copolymers (PLA-GA), or polytetrafluoroethylene (PTFE); and c) administering said encapsulated cell or tissue to the subject in need of treatment via subcutaneous injection or implant, or directly into organs via either direct injection into the substance of the organ or injection through the vascular system of those organs.

In one embodiment, a method for encapsulation of at least one islet cell encapsulated in a microcapsule, comprising the steps of: a) coating at least one islet cell encapsulated in a microcapsule with photoinitiator; b) suspending the at least one coated islet cell encapsulated in a microcapsule in a macromer solution comprised of macromer; and c) irradiating the suspension with light.

In one embodiment, the macromer is a water soluble, ethylenically unsaturated, polymer susceptible to polymerization into water insoluble polymer through interaction of at least two carbon-carbon double bonds.

In one embodiment, the macromer is selected from the group consisting of ethylenically unsaturated derivatives of poly(ethylene oxide) (PEO), poly(ethlyene glycol) (PEG), poly(vinyl alcohol) (PVA), poly(vinylpyrrolidone) (PVP), poly(ethyloxazoline) (PEOX), poly(amino acids), polysaccharides, and proteins.

In one embodiment, the polysaccharides are selected from the group consisting of alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives and carrageenan.

In one embodiment, the proteins are selected from the group consisting of gelatin, collagen, and albumin.

In one embodiment, the photoinitiator is any dye that absorbs light having a frequency between 320 nm and 900 nm, can form free radicals, is at least partially water soluble, and is non-toxic to the at least one islet cell at the concentration used for polymerization.

In one embodiment, the macromer solution further comprises a primary, secondary, tertiary, or quaternary amine cocatalyst and the photoinitiator is selected from the group of ethyl eosin, eosin Y, fluorescein, 2,2-dimethoxy, 2-phenylacetophenone, 2-methyl, 2-phenylacetonphenone, camphorquinone, rose bengal, methylene blue, erythosin, phloxime, thionine, riboflavin, and methyl green.

In one embodiment, the microcapsule is comprised of material selected from the group of alginate, chitosan, agarose, and gelatin.

In one embodiment, the macromer solution further comprises an accelerator to increase the rate of polymerization.

Additional embodiments are described in the following paragraphs.

Paragraph 1. A composition comprising: encapsulating devices comprising a micro-bulk coating, and cell aggregates, wherein said composition has a cell density of at least about 100,000 cells/ml, wherein the micro-bulk coating for the encapsulating devices comprises a polymerizable high density ethylenically unsaturated polyethylene glycol (PEG) having a molecular weight between 900 and 20,000 Daltons, and a sulfonated comonomer, and wherein the micro-bulk coating comprises salt, MOPS (3-(N-morpholino)propanesulfonic) acid, co-monomer, a diol containing compound, an x-ray contrast agent and a photo-initiator.

Paragraph 2. The composition of paragraph 1, wherein the encapsulating devices are micro-bulk capsules.

Paragraph 3. The composition of paragraph 1 where the PEG is selected from the group consisting of a diacrylate of PEG with a molecular weight in the range of 2 kD to 16 kD, a triacrylate of PEG with a molecular weight in the range of 3 kD to 16 kD, a tetra-acrylate of PEG with a molecular weight in the range of 4 kD to 20 kD, and combinations thereof.

Paragraph 4. The composition of paragraph 1 where the co-monomer is selected from the group consisting of AMPS (2-Acrylamido-2-methylpropane sulfonic acid), ammonium AMPS, 2-methyl-2-((1-oxo-2-propenyl)amino)-monoammomium salt, nVP (N-Vinylpyrrolidone), polyvidone, polyvinylpolypyrrolidone and similar types of co-polymers.

Paragraph 5. The composition of paragraph 1 where the x-ray contrast agent is selected from the group consisting of nycodenz, iohexol, omnipaque and similar low-osmolality agents.

Paragraph 6. The composition of paragraph 1 where the salt has the formula of XCl₂(H₂O)ₐ; where X=calcium, magnesium, barium or strontium; and a=0, 1, 2, 4 or 6.

Paragraph 7. The composition of paragraph 1 where the photo-initiator is selected from the group consisting of eosin Y, tetrabromo derivative of fluorescein, methylated eosin Y, ethylated eosin Y, eosin yellowish, bromofluoresceic acid, acid red 87, bromoeosine, eosin B, dibromo dinitro derivative of fluorescein, and similar compounds.

Paragraph 8. The composition of paragraph 1 where the diol containing compound is selected from the group consisting of PEG-diol, beta propylene glycol, propylene-1,3,diol, bisphenol A, 1,4-butanediol and similar compounds.

Paragraph 9. The composition of paragraph 2, wherein the micro-bulk capsule envelopes the cell aggregate.

Paragraph 10. The composition of paragraph 9, wherein the cell aggregate is pancreatic islets.

Paragraph 11. The composition of paragraph 9, wherein the cell density is at least about 6,000,000 cells/ml.

Paragraph 12. The composition of paragraph 1, where the cell is selected from the group consisting of neurologic, cardiovascular, hepatic, endocrine, skin, hematopoietic, immune, neurosecretory, metabolic, systemic, and genetic.

Paragraph 13. The composition of paragraph 12, where the cell is selected from the group consisting of autologous, allogeneic, xenogeneic and genetically-modified.

Paragraph 14. The composition of paragraph 12, where the endocrine cell is an insulin producing cell.

Paragraph 15. A composition comprising a plurality of encapsulating devices having an average diameter of less than 500 μm, said encapsulating devices comprising encapsulated cell aggregates within a micro-bulk coating of an encapsulation material, wherein the composition comprises at least about 500,000 cells/ml and wherein the encapsulation material comprises a polymerizable high density ethylenically unsaturated PEG having a molecular weight of between 900 and 20,000 Daltons, and a sulfonated comonomer, wherein the micro-bulk coating contains the encapsulated cell aggregates.

Paragraph 16. The composition of paragraph 15, wherein the average diameter of the encapsulating device is less than 400 micron.

Paragraph 17. The composition of paragraph 15, wherein the average diameter of the encapsulating device is less than 300 micron.

Paragraph 18. The composition of paragraph 15, wherein the average diameter of the encapsulating device is less than 200 microns.

Paragraph 19. The composition of paragraph 15, wherein the average diameter of the encapsulating device is less than 100 micron.

Paragraph 20. A composition comprising a plurality of micro-bulk encapsulating devices having an average diameter of less than −500 μm, said encapsulating devices comprising encapsulated cells aggregates micro-bulk coated in an encapsulation material, wherein the composition comprises a ratio of volume of encapsulating device to volume of cells of less than about 20:1 and wherein the encapsulation material comprises a polymerizable high density ethylenically unsaturated PEG having a molecular weight between 900 and 20,000 Daltons, and a sulfonated comonomer, and wherein the encapsulation material comprises salt, MOPS (3-(N-morpholino)propanesulfonic) acid, co-monomer, a diol containing compound, an x-ray contrast agent and a photo-initiator.

Paragraph 21. The composition of paragraph 20, wherein the composition comprises a ratio of volume of encapsulating device to volume of cells of less than about 10:1.

Paragraph 22. The composition of any one of paragraphs 1, 15, or 20, where the polymerizable high density ethylenically unsaturated PEG is a high density acrylated PEG.

Paragraph 23. The composition of paragraph 22, where the polymerizable high density acrylated PEG has a molecular weight of 2 kD to 20 kD.

Paragraph 24. The composition of any one of paragraphs 1, 15, or 20, where the sulfonated comonomer is selected from the group consisting of 2-acrylamido-2-methyl-1-propanesulfonic acid, vinylsulfonic acid, 4-styrenesulfonic acid, 3-sulfopropyl acrylate, 3-sulfopropyl methacrylate, and n-vinyl maleimide sulfonate.

Paragraph 25. The composition of paragraph 24, where the sulfonated comonomer is 2-acrylamido-2-methyl-1-propanesulfonic acid.

Paragraph 26. The composition of any one of paragraphs 1, 15, or 20, further comprising a cocatalyst selected from the group consisting of triethanolamine, triethylamine, ethanolamine, N-methyl diethanolamine, N,N-dimethyl benzylamine, dibenzyl amino, N-benzyl ethanolamine, N-isopropyl benzylamine, tetramethyl ethylenediamine, potassium persulfate, tetramethyl ethylenediamine, lysine, omithine, histidine and arginine.

Paragraph 27. The composition of paragraph 26, where the cocatalyst is triethanolamine.

Paragraph 28. The composition of any one of paragraphs 1, 15, or 20, further comprising an accelerator selected from the group consisting of N-vinyl pyrrolidinone, 2-vinyl pyridine, 1-vinyl imidazole, 9-vinyl carbazone, 9-vinyl carbozol, acrylic acid, n-vinylcarpolactam, 2-allyl-2-methyl-1,3-cyclopentane dione, and 2-hydroxyethyl acrylate.

Paragraph 29. The composition of paragraph 28, where the accelerator is N-vinyl pyrrolidinone.

Paragraph 30. A composition comprising encapsulating devices comprising encapsulating cells in an encapsulation material with a polyethylene glycol (PEG) coating having a molecular weight between 900 and 3,000 Daltons, wherein said composition has a cell density of at least about 6,000,000 cells/ml.

Paragraph 31. The composition of paragraph 30, wherein the encapsulating devices are microcapsules.

Paragraph 32. The composition of paragraph 31, wherein the microcapsules are micro-bulk coated cell aggregates.

Paragraph 33. The composition of paragraph 32, wherein the cell aggregates are pancreatic islets.

Paragraph 34. The composition of paragraph 30, where the cell is selected from the group consisting of neurologic, cardiovascular, hepatic, endocrine, skin, hematopoietic, immune, neurosecretory, metabolic, systemic, and genetic.

Paragraph 35. The composition of paragraph 34, where the cell is selected from the group consisting of autologous, allogeneic, xenogeneic and genetically-modified.

Paragraph 36. The composition of paragraph 35 where the endocrine cell is an insulin producing cell.

Paragraph 37. A composition comprising a plurality of encapsulating devices having an average diameter of less than 400 μm, said encapsulating devices comprising encapsulated cells in an encapsulation material, wherein a cell density is at least about 6,000,000 cells/ml.

Paragraph 38. The composition of paragraph 37, wherein the average diameter of the encapsulating device is less than 300 micron.

Paragraph 39. The composition of paragraph 38, wherein the average diameter of the encapsulating device is less than 200 micron.

Paragraph 40. The composition of paragraph 39, wherein the average diameter of the encapsulating device is less than 100 micron.

Paragraph 41. The composition of paragraph 40, wherein the average diameter of the encapsulating device is less than 50 micron.

### A. Background of PEG Interfacial Polymerization for Islets

One of the inventors developed a PEG based islet encapsulation methods and patents that were utilized for their primate studies and their FDA approved clinical trials. This technology is defined in the 2008 U.S. Pat. No. 7,427,415. The clinical trial was closed with partial islet function for greater than one year. The uniqueness of this technology was that islets were not first encapsulated into a matrix that was then was treated for crosslinking and permeability parameters. So this technology was a clear departure from alginate and similar islet hydrogel encapsulation techniques. Instead, each islet was coated with a dye, Eosin Y, which was placed on the surface of the islets. These stained islets were then placed into solution with the PEG encapsulating components. Since the photoinitiator was located in the solution along with other encapsulating ingredients, the radical based crosslinking of the polymer was accomplished by exposure to a high intensity laser beam focused on the bottom of the dish containing the stained islets. A major problem with this approach was the interfacial polymerization on the Y ((polyethylenimine-3-(acrylamidopropyl) trimethyl ammonium chloride. Eosin-5-Isothiocyanate), The other components were combined into new islet capsules using PEG-acrylates NVP (N-vinyl-2-pyrrolidinone), a co-monomer, AMPS (Sodium 2-Acrylomido-2-methyl-1-propansesulfonic acid) solution and also a co-monomer along with other salt solutions using high intensity LED light to cross link the polymetric coating. The new process now permits the formation of a new, minimal volume coating of individual islets that can readily replace the interfacial coatings.

### B. PEG Micro-Bulk Phase, Minimal Volume Capsules Proposed Alternative for Human Islet/iPS/ESC Encapsulation

A significant alternative has been developed to the interfacial polymerized PEG for a diabetes therapy product sourcing for human pancreatic islet cells (Islets)/Induced Pluripotent Stem cell (iPS cell)/Embryonic Stem Cell (ESC's) formed aggregates. PEG acrylate Micro-Bulk phase technology can encapsulate individual human Islets, iPS cell islet aggregates, and/or ESC islet aggregates by minimal volume capsules. Several technical improvements in islet encapsulation enable this new approach. Minimal Volume Capsules of alginate have been developed to reduce standard sizes of alginate capsules from 500+ micron sized capsules containing a single islet to 250-400 micron sized islets that can centralize them within the capsule. By applying the methods used to make these smaller sized alginate capsules to PEG encapsulation, one can now develop “Micro-Bulk” phase islet encapsulation. Micro-Bulk phase encapsulation techniques to encapsulate Islets/IPS cells/ESC's as minimal volume capsules within the very similar PEG coatings. This starts with islets/IPS cells/ESC's being encapsulated in the PEG reactants as small capsules that are then irradiated with LED light to crosslink the PEG surrounding the islets. This reduces the oxygen based radical islet destruction since the eosin Y is no longer on the islet surface in high concentration but is now in the Micro-Bulk phase encapsulation solution at lower concentrations. It also eliminates the requirement to contractually produced dendrimer eosin Y to keep it on the cell aggregate surface for interfacial polymerization. Finally, this approach eliminates the laser requirement due to recent advances in LED technology that can deliver the energy required to cross link the islet containing PEG coatings without the high energy required to cross link islets in a large petri dish. Now each islet can be crosslinked as it moves through the encapsulation device using far less energy as compared to crosslinking in large petri dish sized containers.

### 1. Micro-Bulk Phase Encapsulation Systems for Islets/iPS Cells/ESC's

a. Conversion of the Nisco Encapsulator for Islet Micro-Bulk Phase Encapsulation

Nisco Encapsulator was developed for standard, large-scale alginate encapsulation of cell aggregates utilizing high voltage to reduce islet capsule sizes. This method can also encapsulate islets/iPS cells/ESC's in PEG small capsules that do not require calcium or barium crosslinking. Instead of forming the alginate encapsulated islets and collecting them in a calcium chloride bath for crosslinking, the Nisco Encapsulator can be modified so that the PEG Micro-Bulk phase encapsulation can permit LED illumination following the formation of the minimal volume capsules in PEG.

Just like alginate capsules using this approach, the PEG encapsulated islets are not centralized leading to portions on the edge of capsules inadequately covered as well as high concentrations of empty capsules.

b. Micro-Bulk Encapsulation Using a Tower Apparatus for Formation of the PEG Capsules Containing Islets/iPS Cells/ESC's

This technique is a larger scale alternative to the other approaches as a system that would be more scalable to manufacturing levels. The development of this approach was done by the following three steps:

i. Formation of Micro-Bulk Droplets

The first step was to develop a method in which the droplet formation of the PEG polymer that would include islets/iPC cells/ESC's could be controlled and optimized in terms of a micro-drop size. A small system was built which controlled the flow rate of the fluid containing the PEG polymer exiting a small bore inner needle while simultaneously providing controlled nitrogen gas to flow through a larger outer needle to reduce droplet size. Collection of these PEG droplets initially was into open petri dishes. Flow rates were readily available to slow the formation of polymer droplets that fell into the petri dish containing HBSS. These droplet sizes could be controlled well and when captured underneath focused LED lights of the proper wavelength would readily crosslink.

ii. Optimization of PEG Encapsulation Components

Once this was demonstrated, a series of PEG capsules were produced at a small size as a platform used to optimize the PEG ratios of components required to readily optimize the crosslinking of the capsules. The following components were optimized for capsule size, speed of polymerization, and optimal capsule morphology:

a. PEG acrylate size—from 1.1 kD diacrylate to 10 kD diacrylate and 10 kD tetra-acrylate at different concentrations

b. Optimization of nVP concentrations

c. Optimization of AMPS concentrations

d. Optimization of Nycodenz concentrations

e. Optimization of PEG diol concentrations

Once these optimizations were accomplished, they were combined into a final formulation and method that was tested under a variety of conditions. These results demonstrate the empty Micro-Bulk PEG capsules can clearly and uniformly be crosslinked in a very short time, can lose their surface stickiness, can maintain their shape and size over many days time, and are stable in tissue culture solutions for a few weeks.

Once this was accomplished, we turned to the final optimization of the encapsulation and collection steps. The first step in the tower application optimization was to replace the nitrogen gas with a liquid to drive the PEG polymer solution containing the islets/iPC cells/ESC's down the inner cannula to their exit maintain very small capsule size. With this completed, we turned to developing the collection system after capsule formation since dropping them into the bottom of a petri dish was not an optimizable step as the PEG Micro-Drop capsules readily coalesced prior to their being adequately crosslinked. Instead a collection column was developed that became the site of the LED irradiation step to crosslink the PEG polymer. By controlling the density of the liquid in this reaction chamber, the time to complete crosslinking of the PEG micro-bulk capsules could be completed while still slowly falling through this density-controlled liquid. Thus, the recovery step appeared to be more simple by collecting the Micro-Bulk crosslinked PEG polymer encapsulated islets into HBSS off the bottom of the density gradient in the reaction chamber. Unfortunately, testing with this method proved to not be simple with the PEG-diacrylate capsules continuing to coalesce prior to their being completely crossed-linked by the LED light. So this method of forming and crosslinking Micro-Bulk PEG-diacrylate capsules was also placed on hold until other methods could be evaluated.

c. Micro-Bulk Encapsulation Using Micro-Fluidics Formation of PEG Capsules Containing Islets/IPS Cells/ESC's

The use of micro-fluidics has readily been demonstrated to form small beads that contain cells including islets. Also explored was the potential to cross link PEG-acrylate capsules containing islets/iPS cells/ESC's that can be formed into Micro-Bulk capsules using this micro-fluidics approach of capsule formation. Initially, the capsules well formed with the micro-fluidics that then exited the device to drop into a collection system. But, this exit approach essentially returned us to the Micro-Bulk droplet formation then falling into a collection mode of either a petri dish or a tower in which the time to complete the encapsulation of the PEG-diacrylate was also too long to prevent coalescence of the Micro-Bulk PEG capsules into larger and larger blobs of PEG-diacrylate macro-capsules containing multiple islets. In order to perform the LED induced crosslinking completely within a micro-fluidic may be possible but is beyond our current capabilities. So this approach was also placed on hold.

d. Micro-Bulk Encapsulation Utilizing Emulsion Techniques

An emulsion technique was developed that was required for the encapsulation of the TRGel thermally crosslinked capsules. This technique had to be modified to replace the thermal crosslinking with LED illumination crosslinking of the emulsion produced PEG capsules that can contain islets.

Previous research has demonstrated the feasibility of encapsulating islets by conformal coatings laid down on the surface of islets by binding the photoinitiator to the surface of the islets, followed by laser exposure of light into dish containing the stained islets and all the encapsulation constituents in the media. The crosslinking took place with light activation at the site of the bound eosin Y forming a conformal coating of islets that was self-limiting in thickness of capsule with decreasing crosslinking from the surface to the outer edge of the formed capsules.

The application discloses micro-bulk coating, which is an improved method of encapsulation compared to conformal coating. Emulsion technology was developed to form a micro-bulk coating on the surface of islets. A light box with LED's focused inside achieve high levels of crosslinking which keeps the capsules from fragmenting in vivo. A method was developed to measure fragmentation of capsules. An in vitro capsule testing system was developed to measure the capsule strength in terms of stress and strain prior to implantation. Fragmentation was reduced by increasing concentrations of the micro-bulk components.

Capsule size is an important feature for a successful encapsulation procedure. Reduce Capsule size was reduced by manipulating the encapsulation component conditions.

The following examples are provided merely for illustrative purposes of the present invention and are not to be read as limiting the scope of protection of the present invention.

### EXAMPLES

### Example 1

### PEG Micro-Bulk Encapsulation for Islets

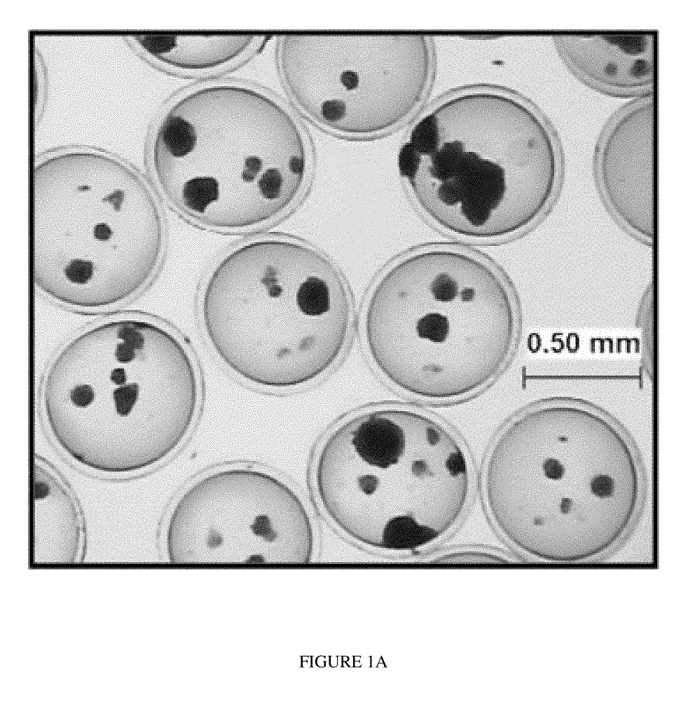

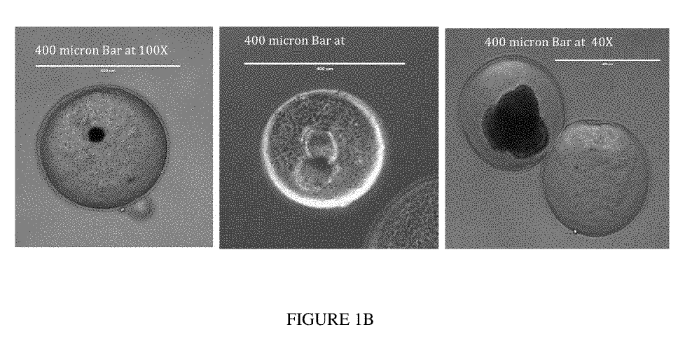

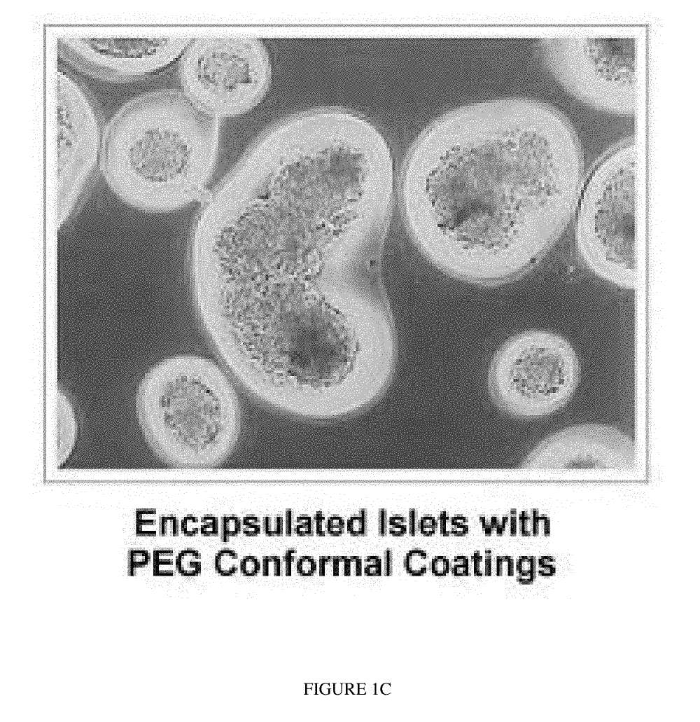

Alginate capsules have been the standard method of isolating islets for over 30 years (FIG. 1A). Their primary problem has been the large size of these capsules relative to the size of the islets that produces implant problems of very large quantities of alginate required for clinical application. While we and others can reduce the size of alginate capsules now and can also increase the numbers of islets per capsule (FIG. 1B), this problem of capsule size remains a critical impediment for considering alginate encapsulation as a way forward to a clinical product. In addition, while highly purified alginates are being formulated, the basic lot-to-lot variations of the seaweed required to produce a standard alginate remains a problem. Conformal coating of islets by PEG tri-acrylate coatings was the best candidate produced in terms of the smallest coating (50-100 microns thick) as well as excellent biocompatibility and functional results (FIG. 1C). It also produced the optimal coatings for long term implant success in diabetic non-human primate implants without insulin or immunosuppression requirements. However, it was lost to further development for human clinical trials due to investor fatigue in supporting the development of a clinical trials and future products that resulted in abandoning its strong patent position.

Volume of PEG conformal coating=20-50 micron coating layer over 150 micron sized islet, then the volume of the conformal coated islet=8×10⁶ um³

If volume of 300 micron capsule of PEG Micro-bulk capsule=14×10⁶ um³ If volume of earlier PEG conformal coating=1, then PEG micro-bulk=1.7 times its volume.

This compares with alginate capsules that usually are 27 times larger if volume of 750 micron diameter alginate capsule is measured with volume=220×10⁶

### Example 2

Initial Implant Results from 1% 1 kDA PEG-Diacrylate Empty Capsules

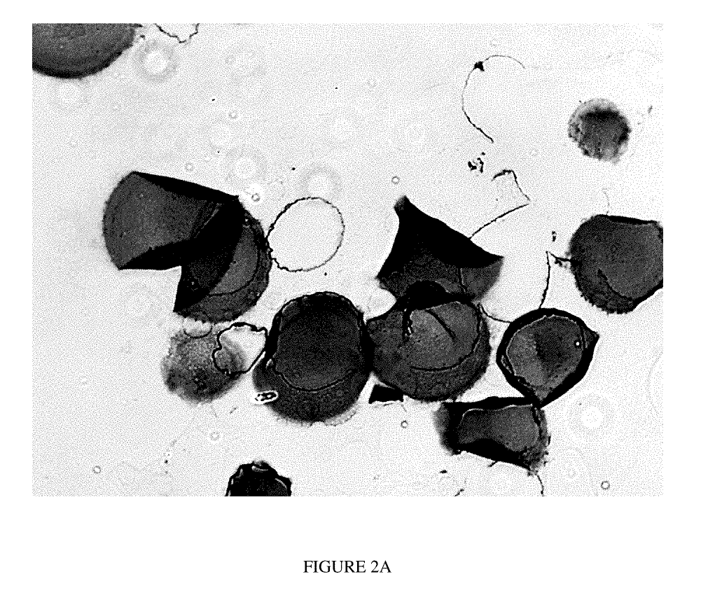

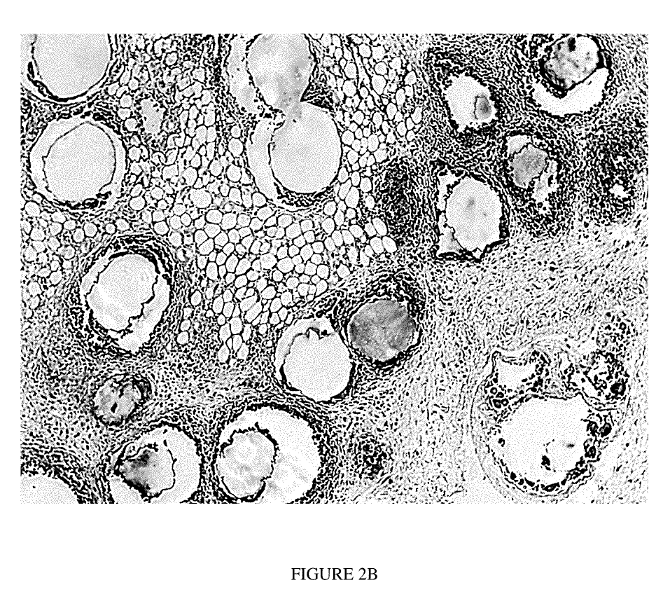

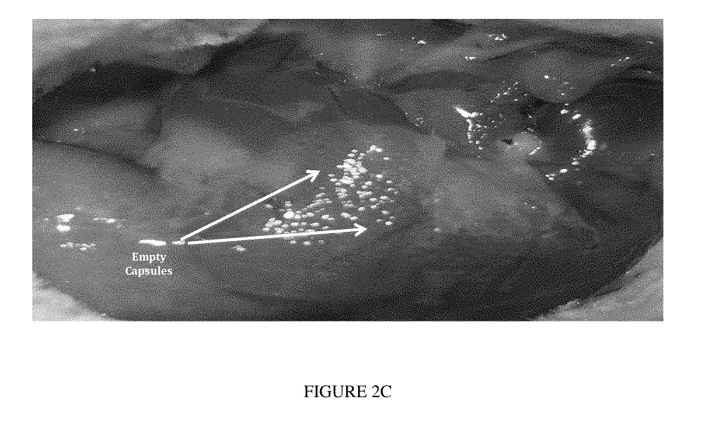

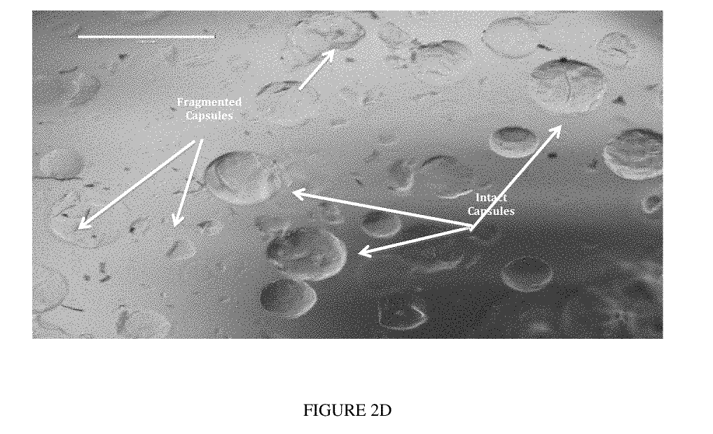

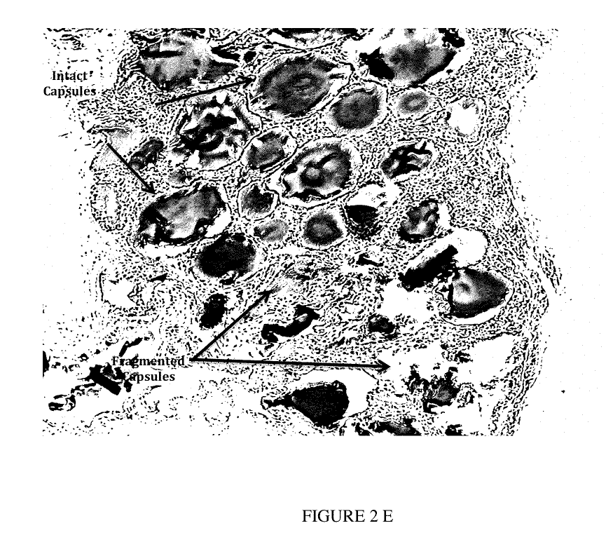

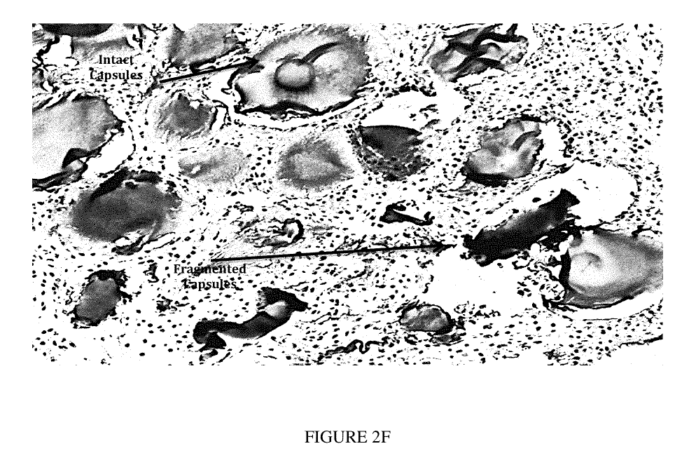

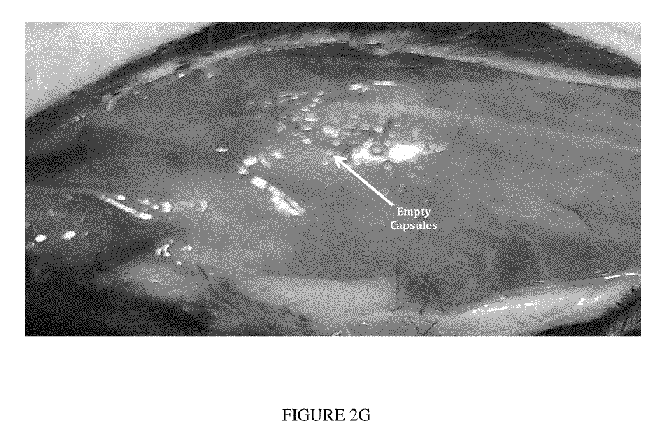

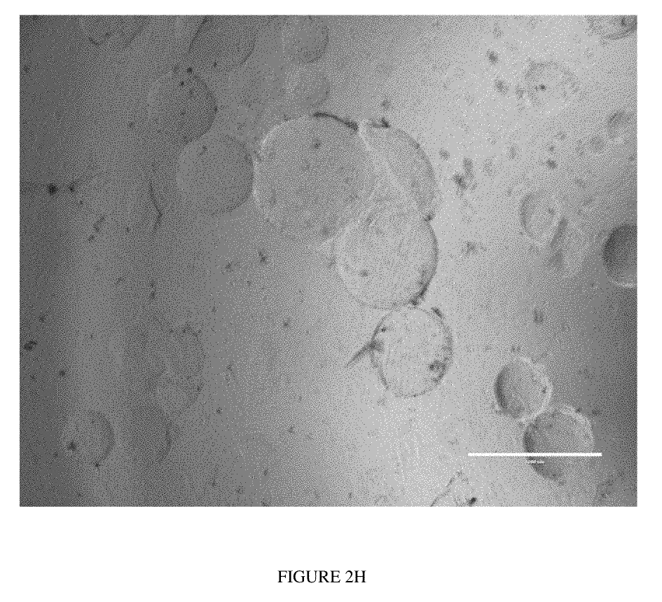

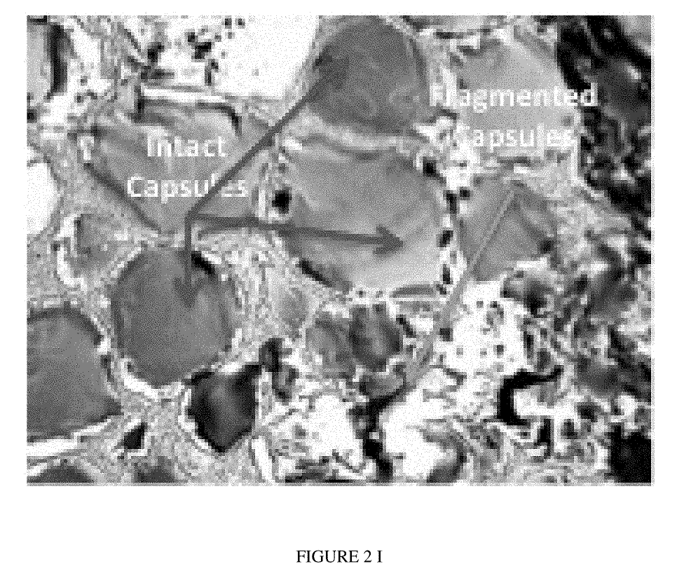

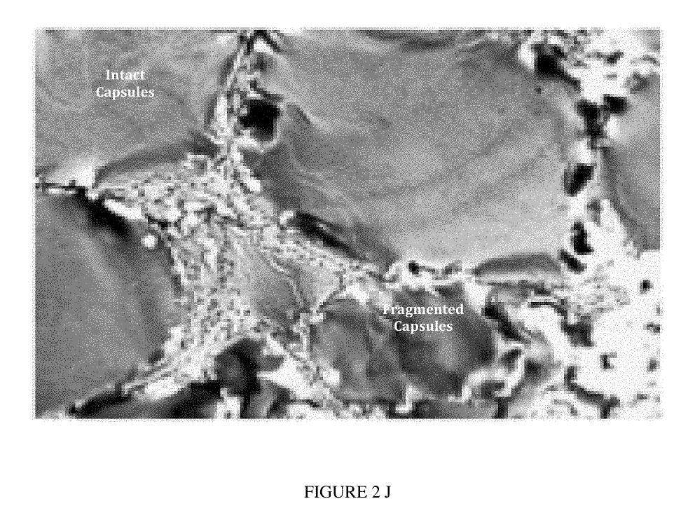

FIGS. 2A & 2B show that the optimized alginate capsules formed with highly purified alginate when formed into empty capsules remain intact two weeks after implant in both Nu/Nu immune incompetent mice and C57Bl6 normal mice. FIGS. 2C & 2D for Nu/Nu implants of 1% 1 kDa PEG Diacrylate in the Nu/Nu mouse recipients demonstrate significant PEG-diacrylate capsule fragmentation after two weeks of implant (FIGS. 2E & 2F). FIGS. 2G & 2H show C57Bl6 implants of 1% 1 kDa PEG-diacrylate also demonstrate significant fragmentation of the empty capsules (FIGS. 2I & 2J).

### Example 3

### Unrestrained Compression Testing for Capsule Strength Testing

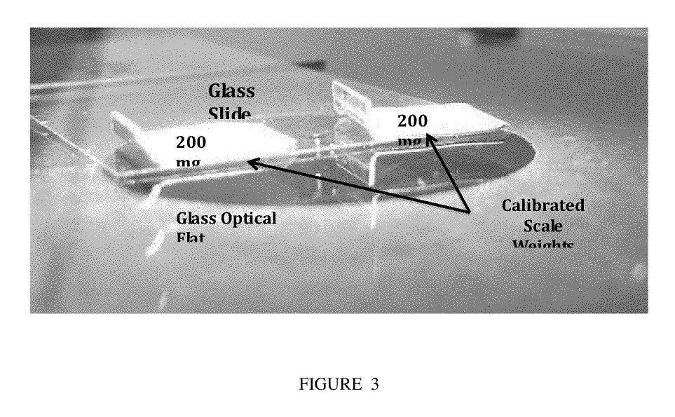

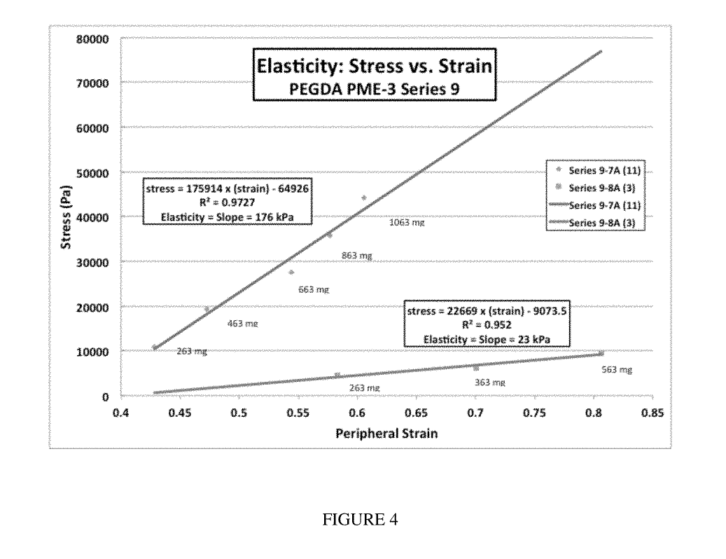

With evidence of capsule fragmentation post-implant, it became obvious that some valid strength testing apparatus had to be developed to initially produce stronger capsules in development and then function as a quality control test post-production prior to implantation. FIG. 3 shows a small apparatus that was developed to place a drop of polymer onto a microscopic glass slide that was then covered by a second glass slide. Specific calibrated weights were sequentially added to document the weight that either broke the polymer bead or reached the maximal weight without breakage. In addition to the weights, Elasticity is actually reported in Pasquelles by calculating by actual measurements of Stress and Strain by scientific definitions. FIG. 4 demonstrates the results of this testing in actual quantification that has been used going forward for all micro-bulk capsules produced.

### Example 4

### Development of PEG Acrylate Micro-Bulk Capsule Size Distribution

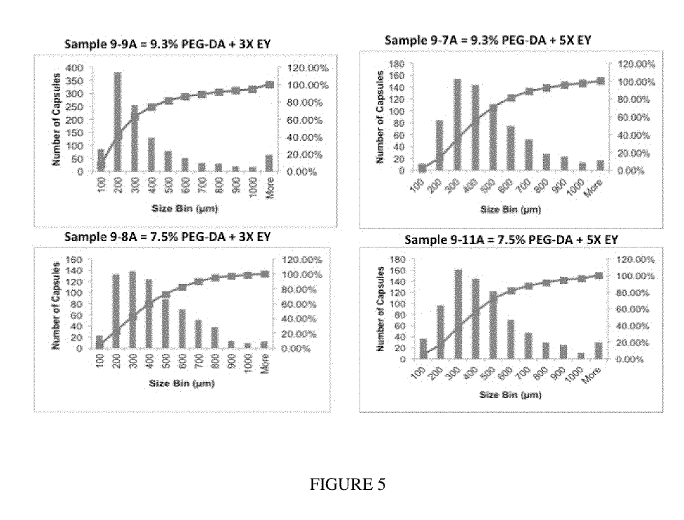

In terms of essential quality control testing, the development of accurate methods to measure islet capsule size distribution of each lot of produced micro-bulk capsules is paramount. To this end, we have developed an improved image analysis system over the one that has been in use for documenting isolated islet size. This new system quantifies islet size by actual calculations and plots the results of the scan in a few minutes as shown in FIG. 5.

### Example 5

### Higher Concentrations of PEG-Diacrylates Prevents In Vivo Capsule Fragmentation

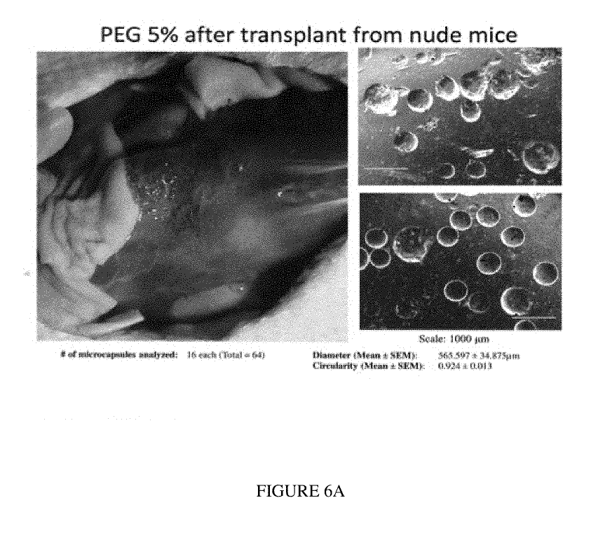

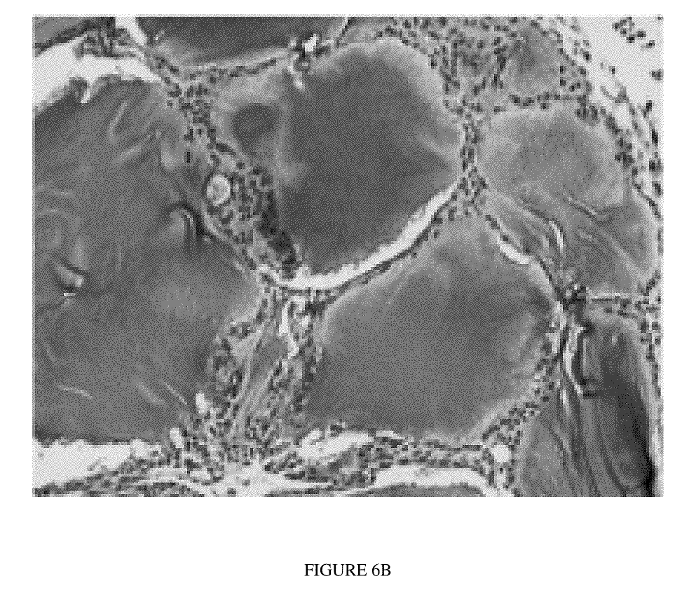

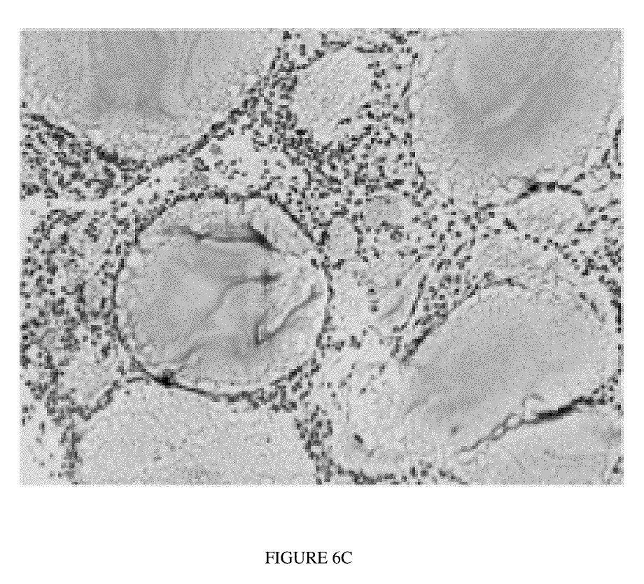

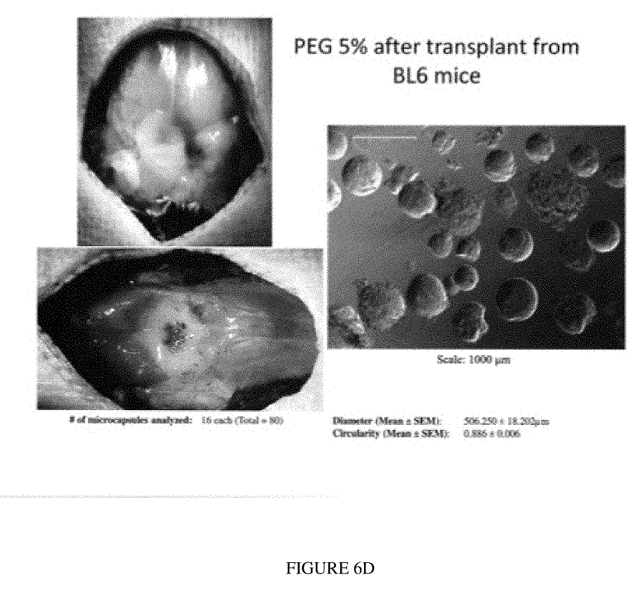

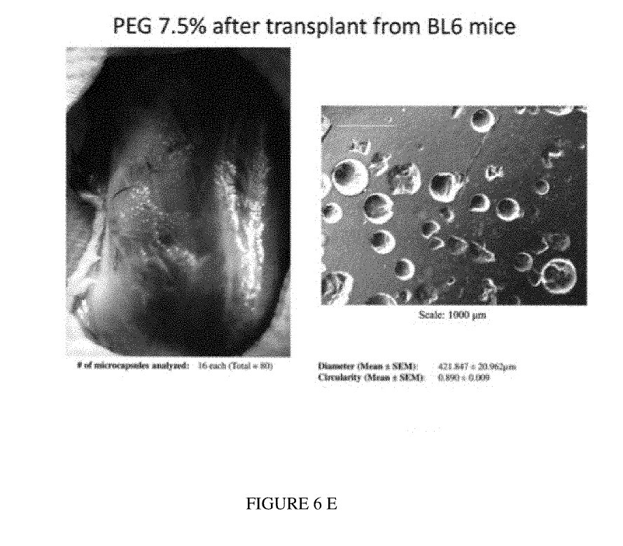

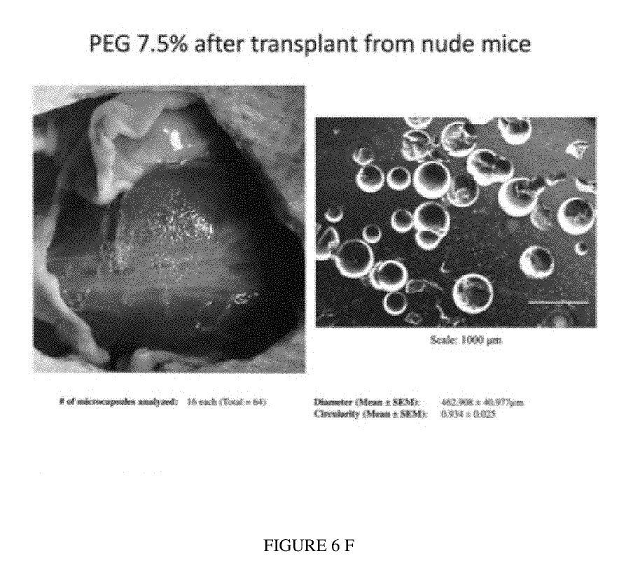

The implants of 5% 1 kDa PEG Diacrylate in both Nu/Nu mice (FIG. 6A) and C57Bl6 (FIGS. 6B & 6C) demonstrate intact capsules without evidence of fragmentation after two weeks of implant. Implants of 7.5% 1 kDa PEG Diacrylate capsules also demonstrate the lack of in vivo capsules at two weeks of implant (FIGS. 6D & 6E). Following implants of 7.5% 1 kDa PEG Diacrylate micro-bulk capsules (FIG. 6F) for longer periods of time demonstrated that these highest concentrations of the polymer became fragile over time eliminating their consideration as a final product. Histologic evidence of the 5% 1 kDa PEG Diacrylate in both the Nu/Nu mice and the C57Bl6 mice demonstrate excellent responses in vivo without evidence of fragmentation.

### Example 6

### Choice of PEG-Acrylates for Micro-Bulk Capsules

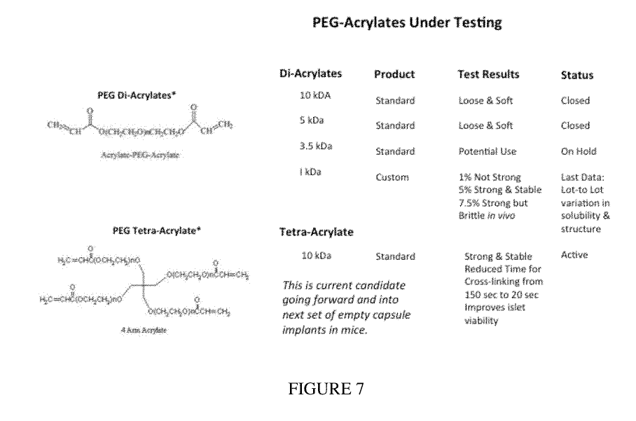

Tetraethylene glycol tri-acrylate (PEG-acrylate) was utilized in the product as its primary encapsulation compound for the production of the confocal encapsulation products but had to be custom manufactured as it is not readily produced under standardized conditions. PEG tri-acrylate is also not listed in the National Center for Biotechnology Information (NCBI) PubChem listing of chemicals. We had determined that the size of the arms is critical for its use in cell encapsulation as the “middle” arm of the three arms is readily hindered by the other two arms in most sizes and applications so there is little room for lot to lot variations in a custom made product. Therefore, we chose to concentrate instead on the PEG-diacrylate and the PEG-tetra-acrylate product candidates to develop for this emulsion based, micro-bulk cell encapsulation product that would greatly reduce the component costs of a final product. FIG. 7 summarizes the results of in vitro testing for micro-bulk islet encapsulation. Starting with the PEG diacrylates, there are three that are readily available (3.5 kDa, 5 kDa, and 10 kDa) by standardized manufacturing and a fourth (1 kDa) that can be obtained by custom manufacturing. The emulsion based micro-bulk capsules produced by both the 5 kDa and the 10 kDa PEG diacrylates produced very soft micro-bulk capsules with a wide range of variable crosslinking from batch to batch and were not tested further. The emulsion produced 3.5 kDA PEG diacrylates were of consistency that could be taken forward as a possible candidate and were placed on hold to complete the testing of these different sized micro-bulk capsule candidates. The emulsion micro-bulk capsules produced by the 1 kDa diacrylate results were concentration dependent. At 1% 1 kDa polymer, the micro-bulk capsules were very soft with little strength. At 5% 1 kDa polymer, these micro-bulk capsules were strong and stable in vitro and appeared stable in the in vivo implants, becoming a potential candidate for final testing. However, as a custom made product, unacceptable lot to lot variations of this difficult to produce PEG diacrylate were encountered due to its very small size and eliminated it as a candidate going forward. At 7.5% 1 kDa PEG diacrylate, the micro-bulk capsules were very strong in vitro, but were found to be very brittle in mouse implants and thus not taken forward. Testing of the 10 kDA four armed PEG tetra-acrylate for in vitro and in vivo testing has been proven now to be the optimal combination of strength and pliability for micro-bulk islet encapsulation. With its 4 arms for reactivity, it has been shown to produce adequately crosslinked micro-bulk capsules with only 20 seconds of LED light exposure compared to the 120 seconds required to achieve adequate crosslinking with the optimized 3.5 kDa PEG diacrylate using the same light exposure. By choosing the 10 kDa PEG tetra-acrylate, we eliminated the problem of encapsulated islet reduced viability from the long, intense and required LED light exposure for the 3.5 kDa PEG diacrylate. Thus, this 10 kDA PEG tetra-acrylate is the final choice to move through the small and large animal pre-clinical studies and on to the clinical trials.

### Example 7

### Elasticity Measurements of Different PEG-Acrylates

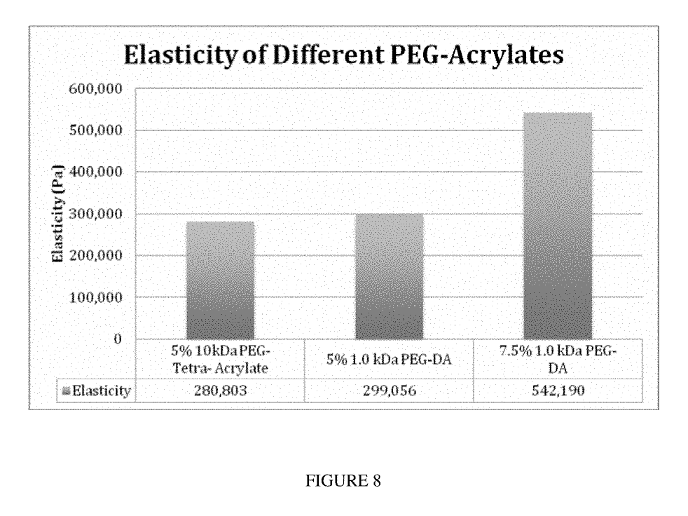

The actual elasticity measurements are shown for the 5% 1 kDa PEG diacrylate and the 7.5% 1 kDa PEG diacrylate showing the increase in measured elasticity parameters of the micro-bulk capsules produced with the higher concentration of PEG-diacrylate concentration (FIG. 8). Since reaching these levels of Elasticity, we have not observed any additional fragmentation in the implanted micro-bulk capsules.

### Example 8

### Improved Encapsulated Islet Viability Using 10 kDa PEG-Tetra-Acrylate Micro-Bulk Capsules

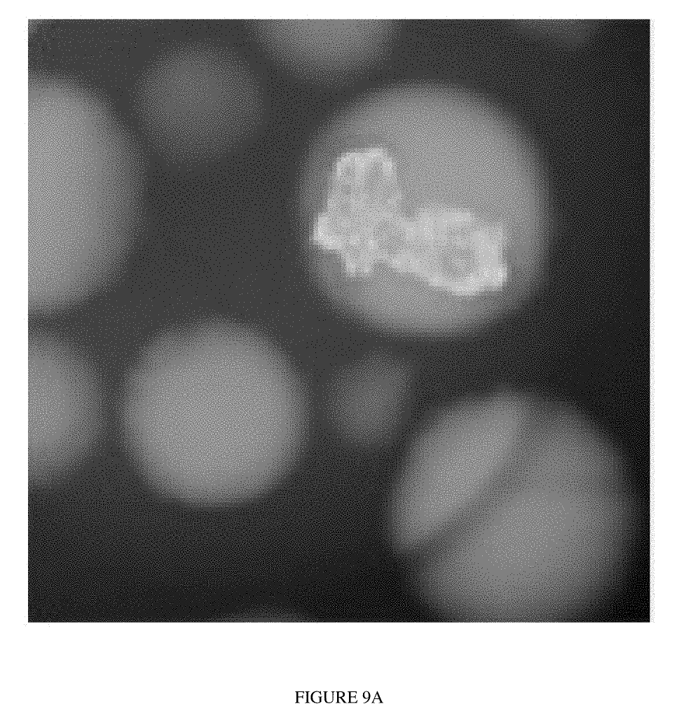

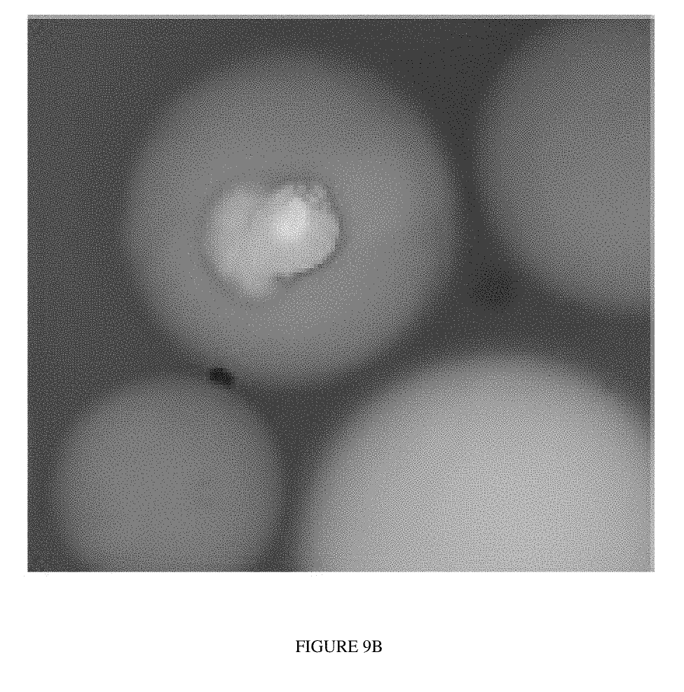

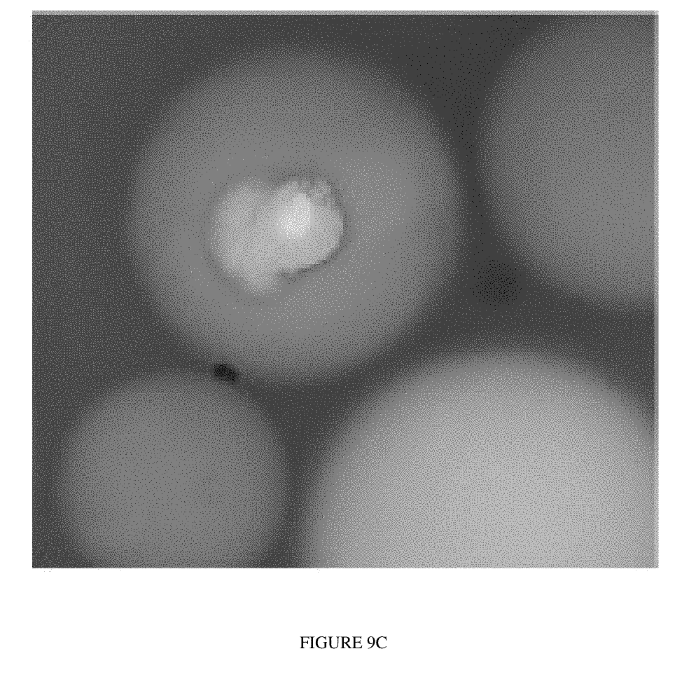

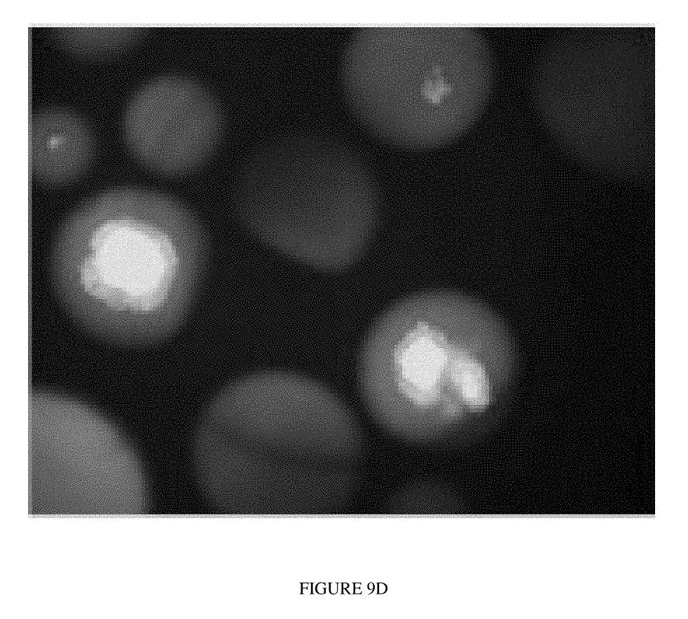

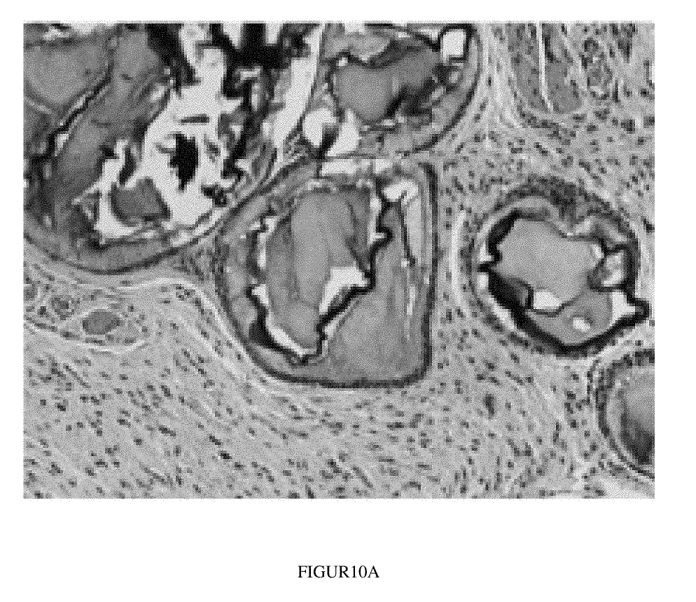

Conversion to the 5% 10 kDa PEG-tetra Acrylate to form the PEG micro-bulk capsules reduced the encapsulation intense light exposure to 20 seconds maintaining excellent encapsulated islet viability. Use of the 5% 1 kDa PEG-Diacrylate (FIGS. 9A & 9B) required 150 seconds to achieve the same degree of crosslinking achieved by the use of the 5% 10 kDa PEG-tetra-Acrylate for only 20 seconds (FIGS. 9C & 9D). But the islet viability was remarkably reduced by the increased light exposure time. So the final polymer choice for the ongoing development of the micro-capsules is clearly the 5% 10 kDa PEG-tetra-Acrylate.

### Example 9

### Successful Implants of PEG Encapsulated Human Islets in Subcutaneous Site

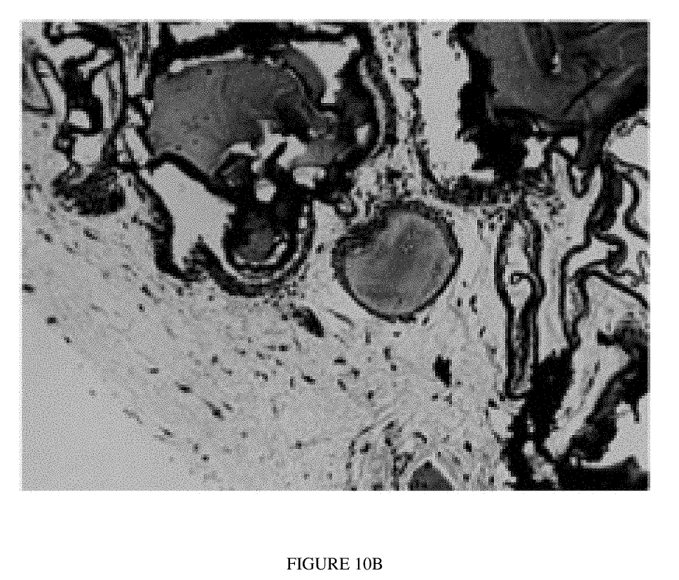

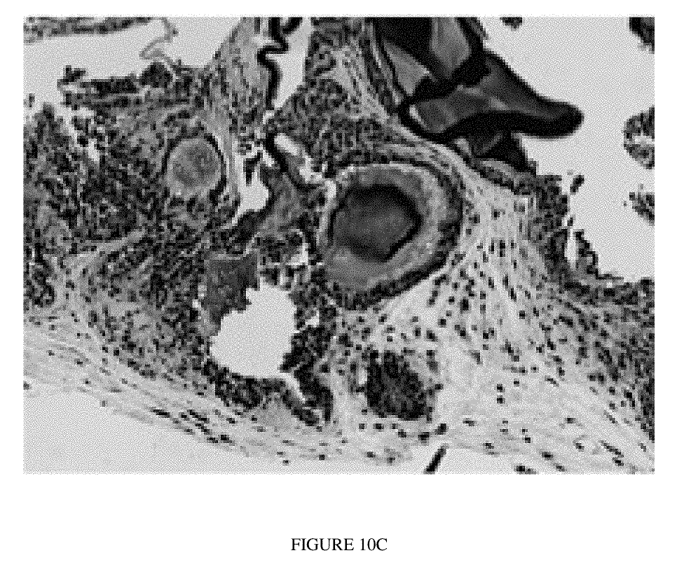

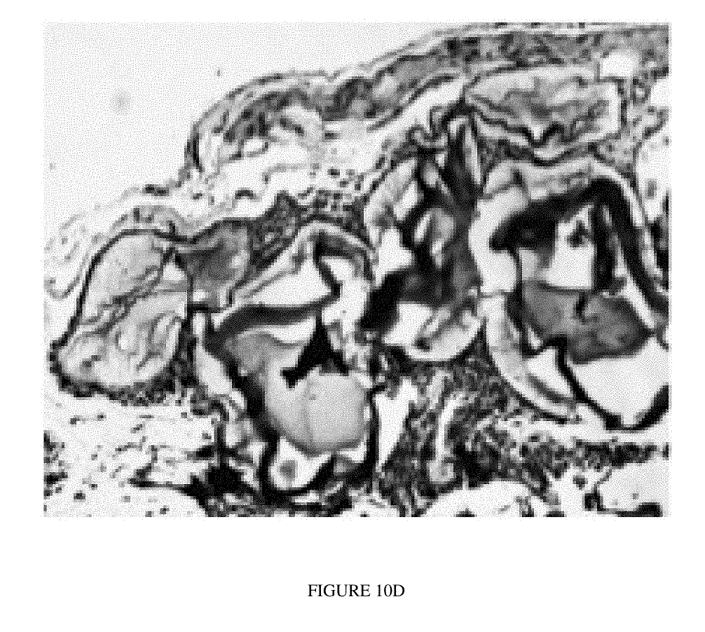

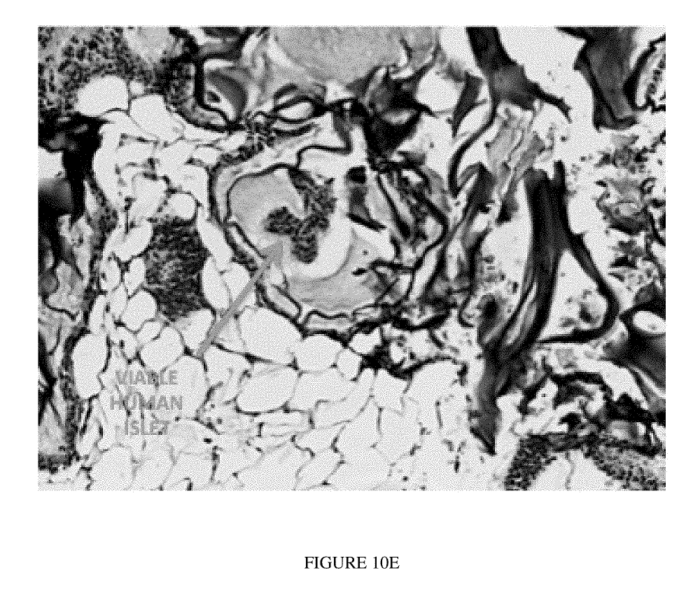

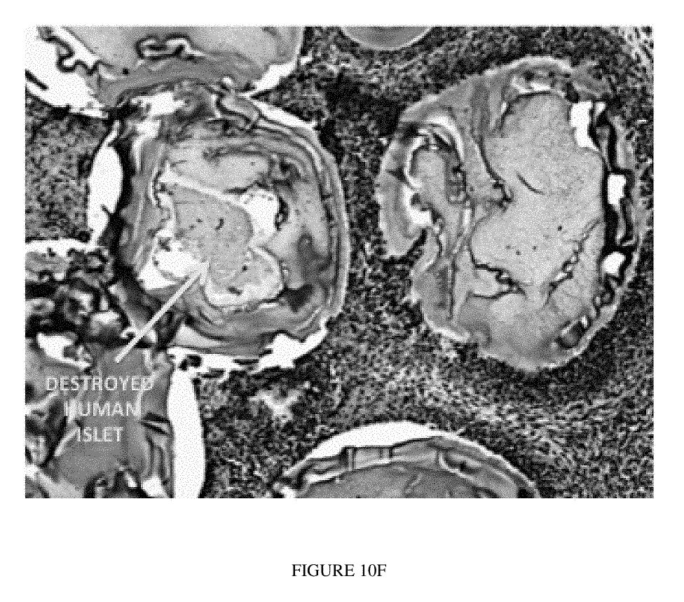

Histologic results from implanting empty micro-bulk 5% 10 kDa PEG-tetra-Acrylate capsules that had previously been tested for elasticity and strength in both the Nu/Nu mice (FIGS. 10A & 10B) and the C57Bl6 mice (FIGS. 10C & 10D) in the subcutaneous site show excellent implants without evidence of fragmentation. There is some evidence of these capsules tending to show some signs of crystallization that is observed as histology knife cutting fractures that are not fragmentation. Human islets were also encapsulated as 5% 10 kDa PEG tetra-acrylate micro-bulk capsules (FIGS. 10E & 10F). After two weeks in culture there is clear evidence of viable islets in both the Nu/Nu mice and C57Bl6. The C57Bl6 recipients show more of a non-specific cellular infiltrate surrounding the human islet containing capsules than was seen with the Nu/Nu mouse recipients that is an expected outcome. These results set the stage to start implanting curative dose of 5% 10 kDa PEG-tetra-acrylate micro-bulk capsules which is the next step.

### Example 10

### Description of Illumination for Photoencapsulation

The encapsulation vessel is made of glass that is optically transmissive in the wavelength band for which photopolymerization is activated. The vessel is contained completely within a chamber made of highly reflective surfaces. Both specular (mirror-like) and diffusing surfaces can be used. The surfaces are arranged to nearly completely enclose the vessel and maximally contain light from sources emitting into the vessel. The surfaces are highly reflective for the emission wavelength band of the light sources which in turn are matched to the active, absorbing wavelengths of the photoactive component of the encapsulation monomer. In one form, these surfaces can be non-reflective for wavelengths that are not useful for stimulating the photoactive component in order to allow loss of light energy from the chamber that is not contributing to photopolymerization.

The chamber has an array of apertures provided. The apertures are optimized in size to allow passage of light from the light source emitters while minimizing loss of light out of the chamber. These apertures can be physical holes or also windows through which the light source wavelengths are effectively transmitted.

The sources are an array of light emitters. In our particular case, these are LEDs. These LEDs have a lens integrated onto the electronic emitter base to provide maximal gathering and directionality of the LED emitted light energy.

The array is geometrically arranged around the outside perimeter of the encapsulation vessel. In our case, there are six emitters spaced at equal intervals around the perimeter. The emitters are placed in close proximity to the encapsulation vessel to maximize light transmission into the vessel. Optical elements such as lenses or wavelength filters may be introduced between the light sources and the vessel to optimize transmission into the encapsulation vessel.

The light sources emit wavelengths selected or adjusted to match as closely as possible the absorption wavelengths of the photoactive component of the macromer. In our case, we have used eosin derivatives with an absorption activity peak near 532 nm and LEDs with an emission peak around 525 nm. This wavelength may be adjustable during the course of photopolymerization in order to optimize the effect of encapsulation.

The positions and power out of the LEDs is adjusted to give a nearly uniform intensity of approximately 120 mW/cm² within the photopolymerization chamber. This intensity may be programmatically adjusted to optimize the photopolymerization process during the time course of encapsulation.

The duration of exposure is controlled to provide illumination for between 15 sec and approximately 250 sec.

The amount of fluorescence emitted by photoactive components within the macromer may be measured during photopolymerization to monitor the progress of the process.

### Example 11

### Micro-Bulk Islet Encapsulation Methodology

The apparatus requires a digital overhead stirrer with a speed range up to 2,000 rpm such as IKA RW 20 Digital Overhead Stirrer that includes the required stirring rods and mixing blades. Heavy glass walled round bottom flasks with a single neck of different sizes for scaling that are used to form the emulsion. The single neck is required in order to enable to spread the emulsion throughout most of the vertical distance of the flask. The Chem Glass CG-1506 fits this requirement along with glass stirring shafts CG-2078 (10 mm) and CG-2087 (19 mm) that attach with Teflon (PTFE) heavy duty stirring blades (CG-2089). One must drill a single 2 mm hole into the glass flask just below the neck for in process chemical additions. The second major apparatus is the LED light box unit that contains a mirrored box with 5 sides, each containing placement of an LED unit that is driven by the appropriate direct current power supplies. The entire apparatus fits into a standard vertical laminar hood equipped with an elevator to lower the base of the unit below the standard hood surface, permitting the apparatus height to all fit within laminar air flow.

The required reagents include cyclonethicone as the water insoluble portion and water based liquids as the soluble portion to form the unstable emulsion by mixing all components contained within the round bottom flask. The water soluble components include the polyethylene glycol acrylate of choice as the primary polymer reactant with co-monomers, MOPS, n-Vinyl pyrrolidione, and AMPS, the photoinitiator eosin Y, Hanks balanced buffer solution, and others. To initiate the formation of the unstable emulsion, one 50 ml conical of cyclomethicone, one of HBSS, and one of water are set aside. The PEG polymer is thawed at 10° C. The round bottom flask is warmed to 37° C. Ambient oxygen is removed by sparging in argon gas from the MOPS and polymer solutions. The sterile flask is positioned beneath the mixer with the mixer shaft and propeller to the bottom of the flask and positioned with the flask and propeller with in the light box with appropriate clamps to secure the apparatus for the high spinning run. Place a few drops of the cyclomethicon within the flask to permit turning on the propeller without friction and set the propeller speed to 1850 rpm. When ready to perform the encapsulation run, ambient lights must be off. To start the run, remove the warm cyclomethicone and add to the flask through the small hole prepared at the top of the flask. With a sterile syringe and needle withdraw air to the 200 μl mark. Take 300 μl of polymer from refrigerator and mix with 20,000 IEQ of human islets within the syringe avoiding any ambient air. Place the needle of the syringe into the small hole in the flask and slowly deliver the islet polymer mixture into the flask. Turn the propeller on to the optimal speed for 30 seconds eliciting the emulsion of the cyclomethicone and the islet/polymer mixture. Activate the LED light bank on for a 20 second exposure of light. Turn on the ambient light and lower the light box below the flask. Remove the propeller from the flask. Decant the oil off the water based liquid containing the micro-bulk encapsulated islets. Slowly fill the flask with HBSS and gently agitate for two minutes and let the product settle. Replace the HBSS with islet culture medium, PIM(R), and rinse twice with additional culture medium. Divide the 20 k micro-bulk islet containing beads evenly into two portions and culture in two non-tissue culture treated T-150 culture flasks with PIM(R) culture media. Then separate small aliquots of the product for the required testing.

### Example 12

### Micro-Bulk PEG Islet Encapsulation

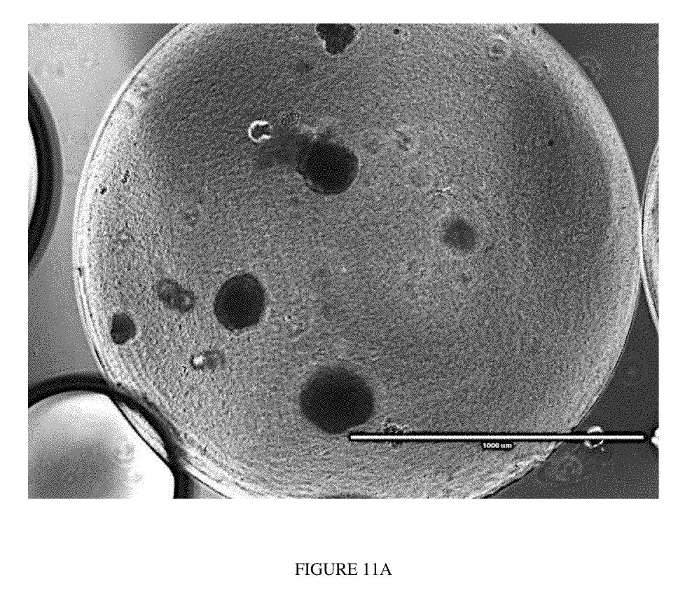

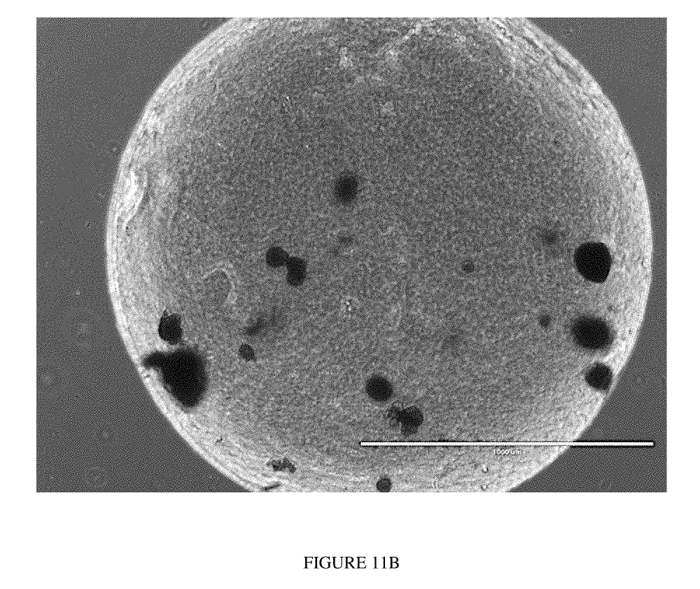

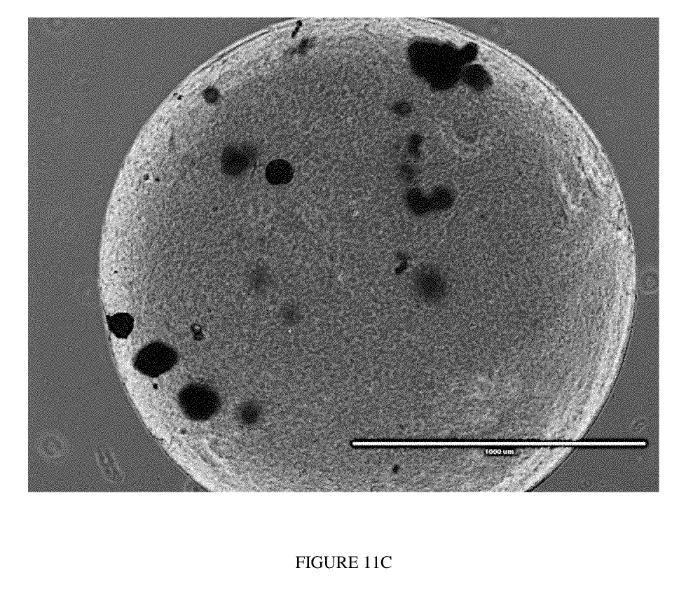

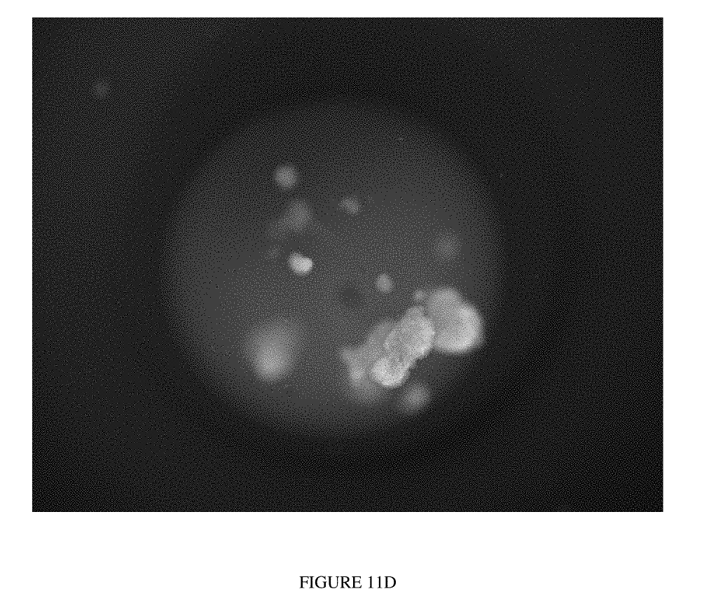

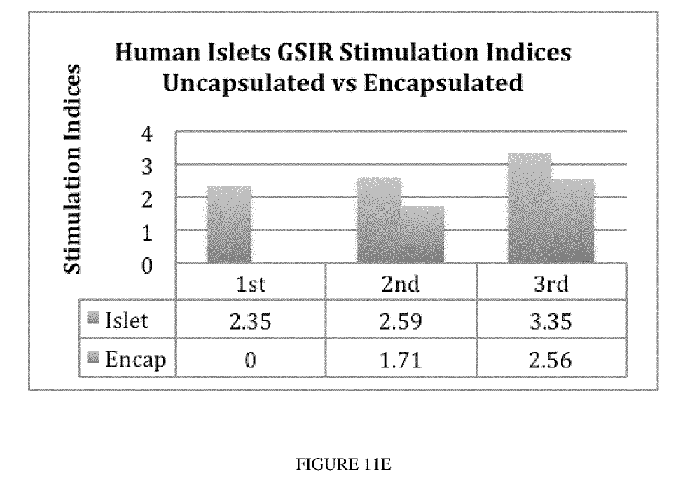

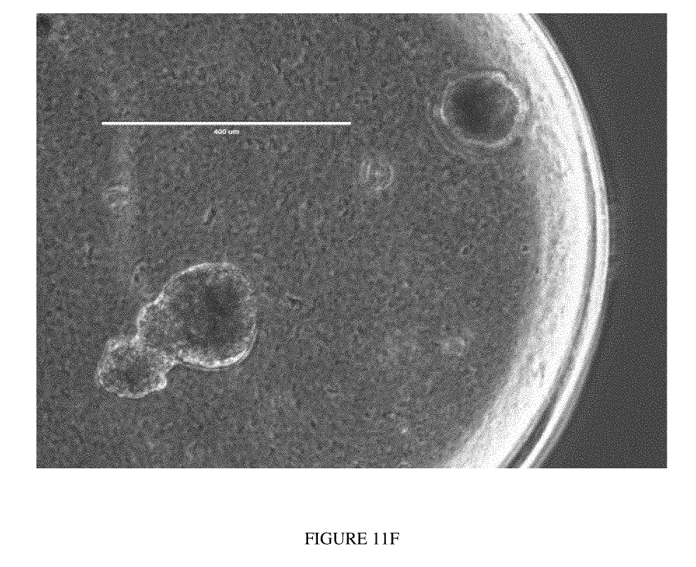

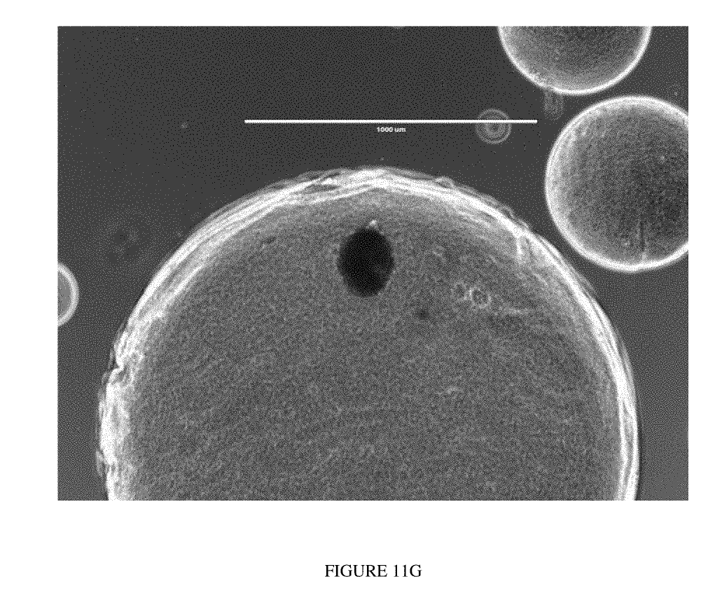

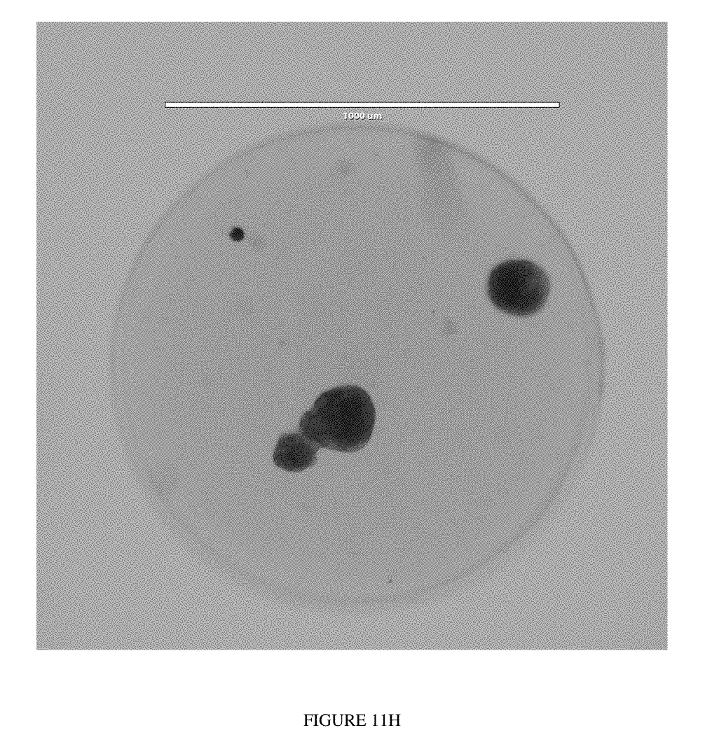

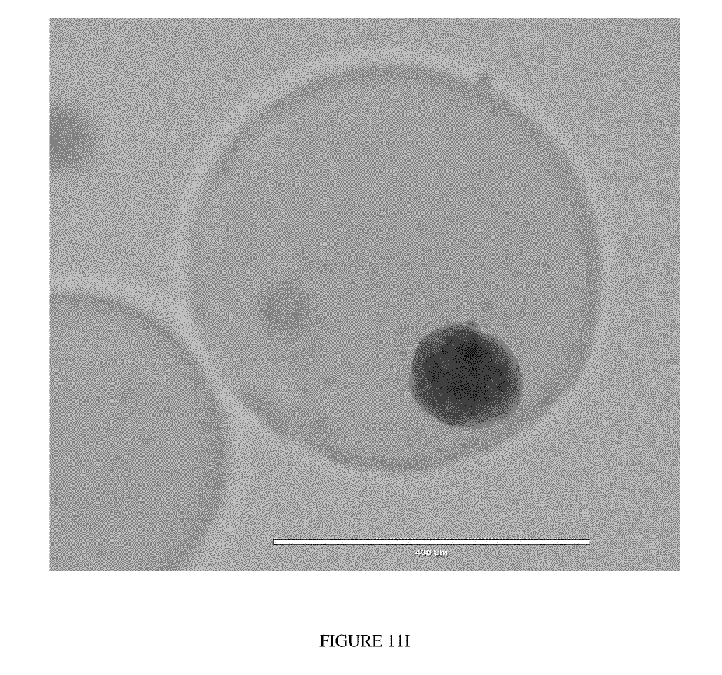

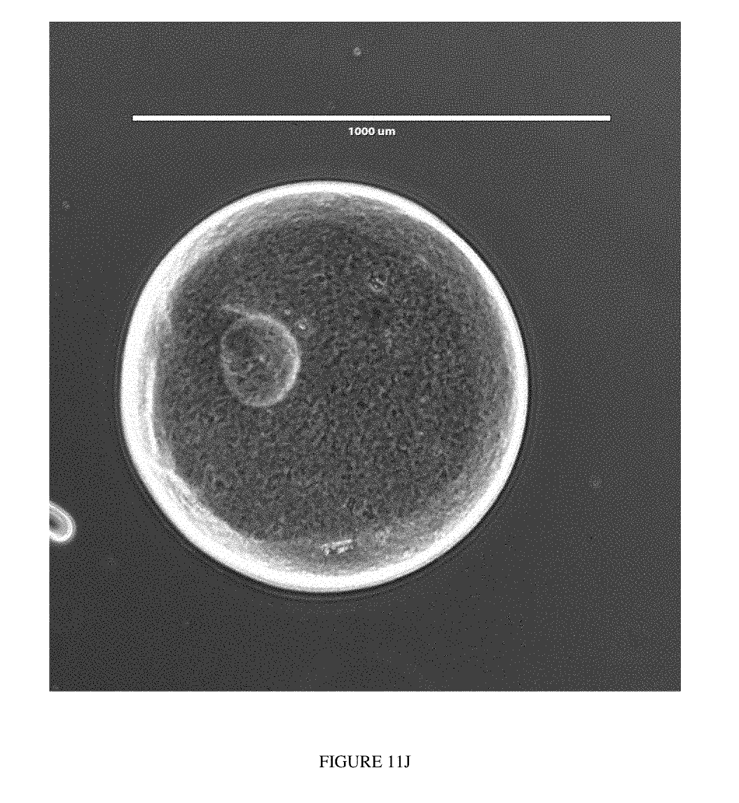

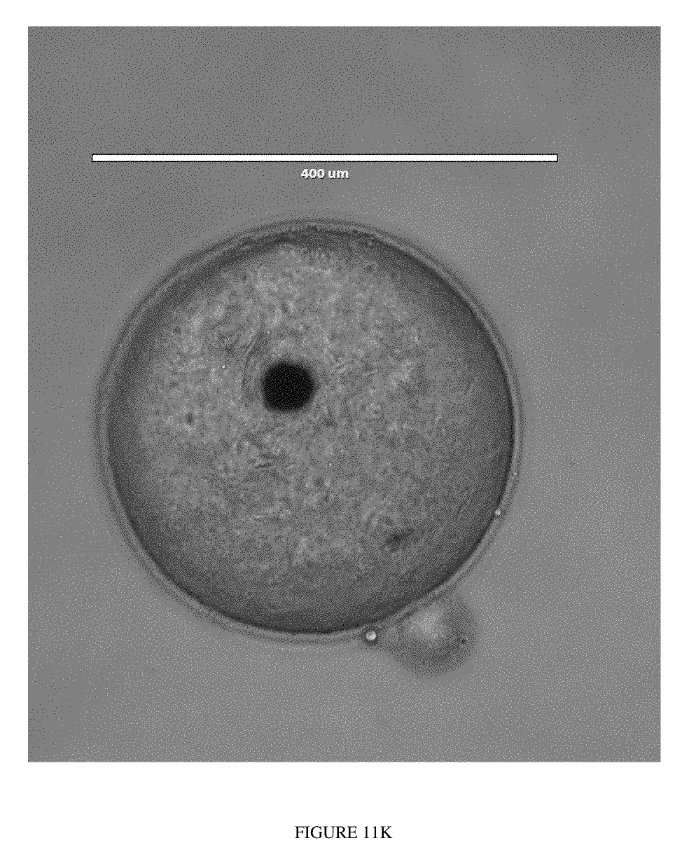

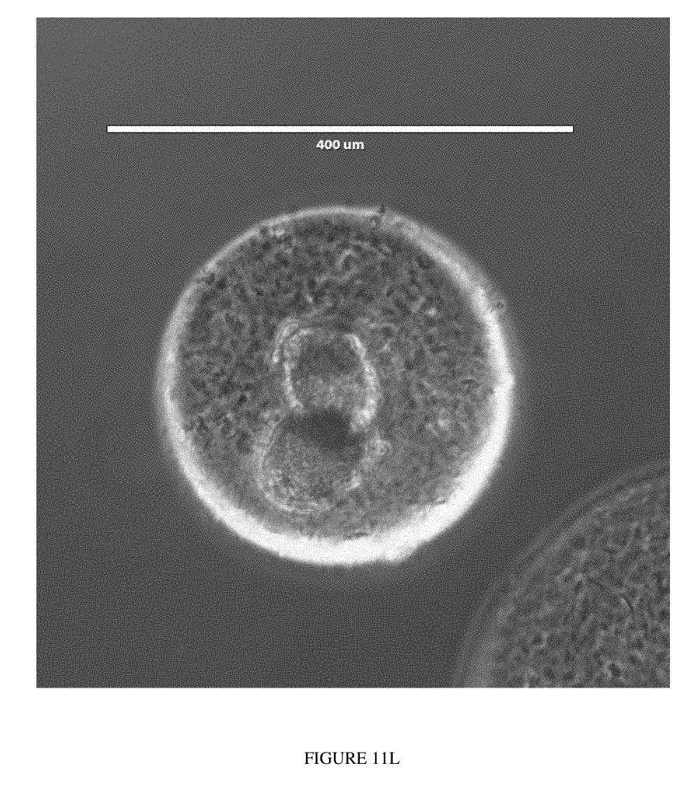

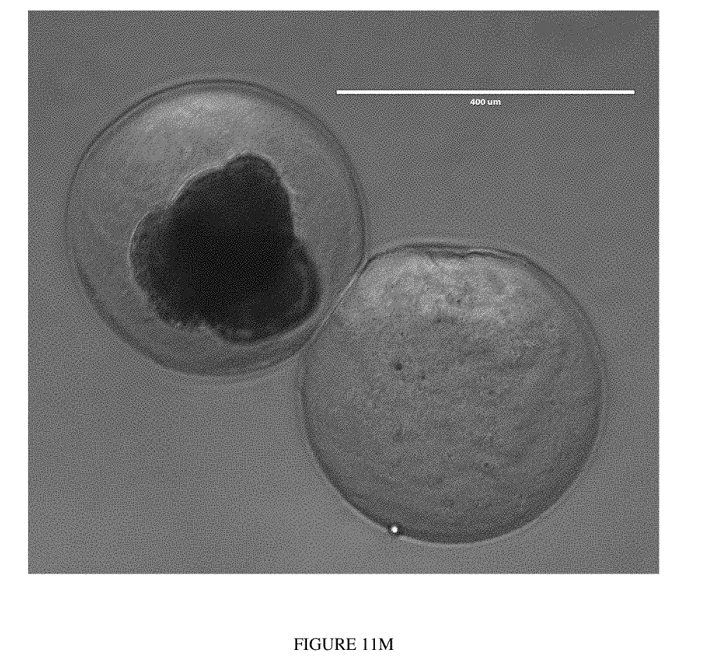

PEG Conformal Coatings have demonstrated that allografts encapsulated islet implanted over two years into a subcutaneous site of diabetic baboons and having initiated a clinical trial of human islet allografts using the same capsules, the inventors developed a second generation of PEG micro-bulk encapsulated islets. FIGS. 11A, 11B & 11C shows the micro-bulk PEG encapsulated human islets along with their viability. These first results were designed to increase the number of human islets per capsule, initially in large capsules, and then reducing the size of these micro-bulk PEG capsules to increase the concentration of human islets per capsule. FIGS. 11A, 11B & 11C show these human islets encapsulated within the micro-bulk PEG capsules are predominantly viable. The in vitro results of glucose stimulation of these micro-bulk encapsulated human islets show their feasibility.

These first large PEG capsules demonstrated that multiple human islets can be encapsulated per capsule to demonstrate the feasibility of this approach for clinical application. Although these first capsules showed the ability to encapsulate multiple human islets per capsule, they were far too large for clinical application. We ran viability tests and GSIR prior to reducing the size of the capsules.

Ethidium bromide/fluorescene diacetate (EB/FDA) staining of the micro-bulk Phase PEG large capsules was performed after one day of culture in PIM(R)® culture medium supplemented with PIM(ABS)® and PIM(G)® (FIG. 11D). The majority of the encapsulated human islets within a single micro-bulk PEG capsule were staining positive (green) and were all completely encapsulated within the large capsule with a few of the smaller islets staining negative (red).

The GSIR results showed the viability of the encapsulated human islets (red) (FIG. 11E). However, compared with the same batch of human islets that were not encapsulated (blue), there is a delay in the encapsulated islets insulin response to increasing glucose. This most likely is a diffusion delay due to these very large PEG capsules and confirm the plans to reduce the size of the micro-bulk PEG capsules for islets.

For the size-reducing studies of the micro-bulk PEG capsules, islet dosing per capsule was reduced. Manipulation of the encapsulation parameters was fairly straight forward using the new device, designed and built specifically for the formation of the micro-bulk PEG capsule

The first step in capsule size reduction was to reduce from >1000 microns to be in the range of 500-1000 microns (FIGS. 11F & 11G). Encapsulated human islets are clearly represented in the photomicrographs as encapsulated within the Micro-Bulk PEG capsules (FIGS. 11H, 11I & 11J).

The reduction from >1000 micron-sized micro-bulk to <500 micron size is critical to providing a clinically relevant encapsulation technology for human islets (FIGS. 11K, 11L & 11M). We increased the number of human islets per capsule as we reduced the size, these early <500 micron sized demonstrated the ability to hold up to three human islets per capsule. Development is ongoing to produce capsules in this range that can routinely hold multiple numbers of islets. It is also critical to have the encapsulated human islets in <400 micron sized capsules since removable rods in implantable devices are around 500 microns in diameter.

It is important to note that the mechanism of crosslinking micro-bulk PEG capsules relies on LED-induced radical polymerization to produce the polymer crosslinking. While this can be very toxic to islets, approaches we utilize during the encapsulation protect the islets from radical damage during the short crosslinking time as demonstrated in the GSIR results. In vivo results of micro-bulk PEG encapsulated human islets are very good.

### Example 13

Implant micro-bulk human islets subcutaneous into diabetic mice with sufficient islets to cure the mice. Monitor their blood glucose levels and add long term insulin support for two weeks so human islets can survive in the subcutaneous site with the temporary insulin support.

### Example 14

Implant micro-bulk human islets subcutaneous into 9 diabetic mice that get 3 implanted with micro-bulk islets in the subQ site, 3 implanted with micro-bulk islets in the IP site, and 3 implanted with free human islets into the IP site.

1. A composition comprising: encapsulating devices comprising a micro-bulk coating, and cell aggregates, wherein said composition has a cell density of at least about 100,000 cells/ml, wherein the micro-bulk coating for the encapsulating devices comprises a polymerizable high density ethylenically unsaturated polyethylene glycol (PEG) having a molecular weight between 900 and 20,000 Daltons, and a sulfonated comonomer, and wherein the micro-bulk coating comprises salt, MOPS (3-(N-morpholino)propanesulfonic) acid, co-monomer, a diol containing compound, an x-ray contrast agent and a photo-initiator.

2. The composition of claim 1, wherein the encapsulating devices are micro-bulk capsules.

3. The composition of claim 1 where the PEG is selected from the group consisting of a diacrylate of PEG with a molecular weight in the range of 2 kD to 16 kD, a triacrylate of PEG with a molecular weight in the range of 3 kD to 16 kD, a tetra-acrylate of PEG with a molecular weight in the range of 4 kD to 20 kD, and combinations thereof.

4. The composition of claim 1 where the co-monomer is selected from the group consisting of AMPS (2-Acrylamido-2-methylpropane sulfonic acid), ammonium AMPS, 2-methyl-2-((1-oxo-2-propenyl)amino)-monoammomium salt, nVP (N-Vinylpyrrolidone), polyvidone, polyvinylpolypyrrolidone and similar types of co-polymers.

5. The composition of claim 1 where the x-ray contrast agent is selected from the group consisting of nycodenz, iohexol, omnipaque and similar low-osmolality agents.

6. The composition of claim 1 where the diol containing compound is selected from the group consisting of PEG-diol, beta propylene glycol, propylene-1,3,diol, bisphenol A, 1,4-butanediol and similar compounds.

7. The composition of claim 2, wherein the micro-bulk capsule envelopes the cell aggregate.

8. The composition of claim 7, wherein the cell aggregate is pancreatic islets.

9. The composition of claim 7, wherein the cell density is at least about 6,000,000 cells/ml.

10. The composition of claim 1, where the cell is selected from the group consisting of neurologic, cardiovascular, hepatic, endocrine, skin, hematopoietic, immune, neurosecretory, metabolic, systemic, and genetic.

11. The composition of claim 10, where the cell is selected from the group consisting of autologous, allogeneic, xenogeneic and genetically-modified.

12. The composition of claim 10, where the endocrine cell is an insulin producing cell.

13. The composition of any one of claim 1, where the polymerizable high density ethylenically unsaturated PEG is a high density acrylated PEG.

14. The composition of claim 13, where the polymerizable high density acrylated PEG has a molecular weight of 2 kD to 20 kD.

15. The composition claim 1, further comprising a cocatalyst selected from the group consisting of triethanolamine, triethylamine, ethanolamine, N-methyl diethanolamine, N,N-dimethyl benzylamine, dibenzyl amino, N-benzyl ethanolamine, N-isopropyl benzylamine, tetramethyl ethylenediamine, potassium persulfate, tetramethyl ethylenediamine, lysine, omithine, histidine and arginine.

16. The composition of claim 15, where the cocatalyst is triethanolamine.

17. The composition of claim 1, further comprising an accelerator selected from the group consisting of N-vinyl pyrrolidinone, 2-vinyl pyridine, 1-vinyl imidazole, 9-vinyl carbazone, 9-vinyl carbozol, acrylic acid, n-vinylcarpolactam, 2-allyl-2-methyl-1,3-cyclopentane dione, and 2-hydroxyethyl acrylate.

18. The composition of claim 17, where the accelerator is N-vinyl pyrrolidinone.
